(19) United States
(12) Patent Application Publication
(10) Pub. No.: US 2020/0038484 A1
(43) Pub. Date: Feb. 6, 2020

(54) Bioconjugation Methods for Targeted in Situ Therapeutic Delivery

(71) Applicants: The Board of Trustees of the Leland Stanford Junior University, Stanford, CA (US); The U.S. Government Represented by The Department of Veterans Affairs, Washington, DC (US)

(72) Inventors: David Myung, San Jose, CA (US); Gabriella Fernandes-Cunha, Palo Alto, CA (US); Hyung Jong Lee, Cupertino, CA (US)

(21) Appl. No.: 16/482,560

(22) PCT Filed: Feb. 5, 2018
(86) PCT No.: PCT/US18/16791

§ 371 Date: Jul. 31, 2019

(51) Int. Cl. A61K 38/18; A61K 35/28; A61K 47/60; A61K 47/64; A61K 47/69

(52) U.S. Cl. CPC A61K 38/1808; A61K 35/28; A61K 38/185; A61K 9/0048; A61K 47/6435; A61K 47/6903; A61K 47/60

(57) ABSTRACT

Bioconjugation methods for promoting wound healing are disclosed. In particular, the invention relates to the in situ application of non-photochemical crosslinking techniques such as copper-free click chemistry using strain-promoted azide-alkyne cycloaddition (SPAAC) or multi-functional succinimidyl esters as a therapeutic delivery modality for biomolecules and stem cells to enhance wound healing.

### TECHNICAL FIELD

The present invention pertains generally to bioconjugation methods for localized delivery of therapeutic factors to human tissue. In particular, the invention relates to the in situ application of non-photochemical crosslinking techniques such as copper-free click chemistry using strain-promoted azide-alkyne cycloaddition (SPAAC) or multi-functional succinimidyl esters to deliver therapeutic biomolecules and stem cells to human tissue.

### BACKGROUND

The following discussion of the background art is intended to facilitate an understanding of the present invention only. The discussion is not an acknowledgement or admission that any of the material referred to is or was part of the common general knowledge as of the priority date of the application.

The wound healing response is limited or impaired in many conditions, such as in diabetic ulcers, burns, chemical exposure injuries, neurotropic keratopathy, and nerve damage. Cell-based therapies such as stem cell transplantation typically provide only cells without the required matrix upon which to grow, or without the stimulatory factors to which to respond by migration, proliferation, and/or differentiation. Topical approaches to wound healing have been reported using epidermal growth factor, thymosin beta 4, nerve growth factor, substance P and insulin-like growth factor, and fibronectin. However, a clinically proven biopharmacologic therapy has not yet been successfully developed.

The corneal epithelium protects the eye against pathogens and plays an essential role in preserving optical clarity. It is damaged in numerous debilitating conditions ranging from severe dry eyes and chemical injury, to corneal ulcers and melts. Loss of the epithelial barrier is the inciting event that results in vision loss in nearly every blinding ocular surface condition, and there are no effective treatments available that specifically promote its repair.

Neurotrophic keratopathy (NK) is a degenerative disease of the cornea resulting from trigeminal nerve damage caused by a variety of conditions including diabetes, herpes, neoplasms, or trauma (Bonini et al. (2003) Eye 17(8):989-995; Dunn et al. (2010) Ann. N.Y. Acad. Sci. 1194(1):199-206). It is hallmarked by decreased corneal sensitivity, reduced reflex tearing, and poor wound healing, leaving the cornea susceptible to injury and progressive breakdown (Bonini et al., supra; Dunn et al., supra). NK poses a particularly difficult clinical challenge due to the limited efficacy of current treatments such as frequent lubrication, antibiotic drops or ointment, patching, and bandage contact lenses. In refractory cases, oral doxycycline, autologous serum, and application of an amniotic membrane, a flap of conjunctival tissue, or tarsorraphy are used alone or in combination (Abelson et al. (2014) Thoughts on Healing the Wounded Cornea, Review of Ophthalmology 2014; September:52-54). Amniotic membranes in particular have shown promising results, but wound closure times are still reported to be two weeks or greater (Kruse et al. (1999) Ophthalmology 106(8):1504-1511; Chen et al. (2000) Br. J. Ophthalmol. 84(8):826-833). Despite the arsenal of modalities available, a protracted clinical course is often required and the healing response can be erratic (Abelson et al., supra), leaving the cornea at risk of infection, scarring, perforation, and blindness (Abelson et al., supra; Nagano et al. (2003) Invest. Ophthalmol. Vis. Sci. 44(9):3810-3815).

Corneal epithelial health is modulated by endogenous neuropeptides supplied by corneal nerves (Bonini et al. (2003) Eye 17(8):989-995). Promising yet limited results have been reported on the therapeutic potential of various topically applied neuropeptides and growth factors (Bonini et al., supra; Dunn et al., supra; Nagano et al., supra; Bonini et al. (2000) Ophthalmology 107(7):1347-1351). For instance, exogenous application of the neuropeptide Substance P (SP) has been shown to improve wound healing in NK, but its effects are enhanced when combined with another trophic agent such as epidermal growth factor (Guaiquil et al. (2014) Proc. Natl. Acad. Sci. USA 111(48):17272-17277). Topical neuroregenerative ligands such as nerve growth factor (NGF) have been shown in clinical trials to restore corneal innervation (Aloe et al. (2008) Pharmacological Research 57(4):253-258; Guaiquil et al., supra), but treatment requires four times daily administration and anywhere from 9 days to 6 weeks for wound closure to occur (Aloe et al. (2012) J. Transl. Med. 10:239). Recently, vascular endothelial growth factor (VEGF) has been shown in an animal model to stimulate regeneration of injured corneal nerves (Guaiquil et al., supra), but these results have not yet been reported in humans. Thus, to date, a clinically available, rapid-onset biopharmacologic therapy for NK remains elusive.

Despite their promise, topical growth factors are limited in effect in part because of their local depletion via endocytosis of growth factor-receptor complexes (Lee et al. (2011) J R Soc Interface 8(55):153-170; Schultz et al. (2009) Wound Repair and Regeneration 17(2):153-162) as well as through substantial volumetric losses into the lacrimal system and by overflow onto the outer lids and cheek. EGF, for instance, has been studied extensively (Pastor et al. (1992) Cornea 11(4):311-314), but has not been clinically successful. NGF has recently been shown in clinical trials to restore corneal innervation, but requires frequent administration and up to 6 weeks for wound closure to occur (Aloe et al. (2012) J Transl Med 10:239-5876-10-239). To date, a rapid-onset biopharmacologic therapy to promote corneal wound healing remains elusive.

Thus, there remains a need in the art for better ways to stimulate a regenerative response in order to foster wound healing and restore anatomy and, in turn, tissue functions such as epithelial barrier effects and neural transmission.

### SUMMARY

The present invention relates to the use of biocompatible bioconjugation methods in situ such as copper-free click chemistry using strain-promoted azide-alkyne cycloaddition (SPAAC) or multi-functional succinimidyl esters to deliver therapeutic biomolecules and/or stem cells to human tissue. In situ bioconjugation can be used in a variety of ways to promote wound healing, including for (i) production of an in situ-forming, growth factor-eluting gel membrane that covers wounds, (ii) direct covalent linkage of growth factors to damaged tissue, and (iii) encapsulation of stem cells in a biocompatible carrier matrix at the surface of damaged tissue. In particular, these methods can be used to stimulate rapid re-epithelialization and nerve regeneration in damaged tissue.

In one aspect, the invention includes a method of treating damaged tissue in a subject, the method comprising: a) contacting the damaged tissue with effective amounts of one or more growth factors capable of promoting tissue regeneration or repair; and b) crosslinking the one or more growth factors to the damaged tissue using a biocompatible non-photochemical bioconjugation method. Damage to the tissue may be caused, for example, by physical trauma, chemical injury, surgery, or a disease. In certain embodiments, the method further comprises preparing the damaged tissue prior to treating the subject by exfoliation or debridement of fibrotic or necrotic tissue. Exemplary growth factors that can be used in the practice of the invention include epidermal growth factor (EGF), nerve growth factor (NGF), vascular endothelial growth factor (VEGF), and insulin-like growth factor (IGF). In another embodiment, at least two growth factors are crosslinked to the damaged tissue. For example, EGF in combination with NGF can be crosslinked to the damaged tissue, wherein EGF stimulates re-epithelialization and NGF stimulates nerve regeneration in the damaged tissue.

In one embodiment, the invention includes a method of treating damaged tissue in a subject, the method comprising: a) contacting the damaged tissue with effective amounts of one or more conjugated growth factors capable of promoting tissue regeneration or repair; and b) performing strain-promoted azide-alkyne cycloaddition (SPAAC) click chemistry, whereby the one or more growth factors are crosslinked to the damaged tissue.

In another embodiment, the invention includes a method of treating damaged tissue in a subject, the method comprising: mixing at the point-of-care, an extracellular matrix biomolecule (e.g., collagen, laminin, or fibronectin), a growth factor, and a multi-functional succinimidyl ester of polyethylene glycol (PEG) that acts as a crosslinking agent to form a gel that entraps the growth factor within the hybrid collagen-PEG matrix. Other multi-functional succinimidyl esters can be used as well.

In certain embodiments, more than one bioconjugation step is performed. For example, at least one bioconjugation step may be performed in vitro, and at least one bioconjugation step may be performed directly on the damaged tissue. SPAAC click chemistry can be used to crosslink different molecules to a tissue surface. For instance, either one or a plurality of different growth factors can be conjugated with an azide or strained alkyne moiety, while the tissue surface can be conjugated with the counterpart azide or strained alkyne. As will be clear to one of skill in the art, some bioconjugation chemistries are more advantageous depending on the conditions, the part of the body being treated, and the particular biomolecules, polymers, or other factors being crosslinked. Alternatively or additionally, more than one bioconjugation technique may be used for crosslinking. For example, thiol-ene click chemistry may be used, which provides a different/orthogonal crosslinking methodology than SPAAC. In some embodiments, one or more biomolecules are crosslinked by thiol-ene click chemistry and one or more other biomolecules are crosslinked by SPAAC click chemistry. In some embodiments, the different crosslinking methods are performed simultaneously.

In certain embodiments, bioconjugation comprises performing SPAAC click chemistry. In one embodiment, performing SPAAC click chemistry comprises: a) reacting the damaged tissue with a heterobifunctional azide-N-hydroxysuccinimide (NHS) crosslinker (with or without a spacer arm) to produce azide-derivatized tissue, wherein collagen in the damaged tissue is covalently coupled to a plurality of azide functional groups; b) reacting the one or more growth factors with an alkyne-NHS crosslinker to produce alkyne-conjugated growth factors; and c) reacting the alkyne-conjugated growth factors with the azide-derivatized tissue such that the growth factors are covalently coupled to the damaged tissue.

In another embodiment, performing SPAAC click chemistry comprises: a) reacting the damaged tissue with an alkyne-NHS crosslinker to produce alkyne-derivatized tissue, wherein collagen in the damaged tissue is covalently coupled to a plurality of alkyne functional groups; b) reacting the one or more growth factors with an azide-NHS crosslinker (with or without a spacer arm) to produce azide-conjugated growth factors; and c) reacting the azide-conjugated growth factors with the alkyne-derivatized tissue such that the growth factors are covalently coupled to the damaged tissue.

In another embodiment, the alkyne-NHS crosslinker is selected from the group consisting of dibenzycyclooctyne-N-hydroxysuccinimide (DBCO-NHS), bicyclononyne-N-hydroxysuccinimide (BCN-NHS), dibenzocyclooctyne-sulfo-N-hydroxysuccinimide (DBCO-sulfo-NHS), and their derivatives, such as derivatives that contain a PEG spacer arm of various lengths. The PEG spacer arms may range from one PEG unit to many PEG units in length up to an average molecular weight of about 14,000 Da. The spacer arm may also comprise other types of chemical backbones, such as an aliphatic backbone.

In another embodiment, the azide-NHS crosslinker is an azide-polyethylene glycol (PEG)-NHS crosslinker. PEG polymers of various lengths (azide-PEGₙ-NHS) can be used to alter the spacing between the NHS and azide moieties. The PEG spacer arms may range from one PEG unit to many PEG units in length up to an average molecular weight of about 14,000 Da. The spacer arm may also comprise other types of chemical backbones, such as an aliphatic backbone.

In another embodiment, the method further comprises increasing crosslinking of the growth factors on the tissue using a multi-arm PEG linker comprising azide or alkyne group end-functionality. Exemplary multi-arm PEG linkers include 3-arm PEG, 4-arm PEG, 6-arm PEG, and 8-arm PEG, as well as branched dendrimers of PEG. This could be mixed with one or more biomolecules having the counterpart functionality, effectively creating a hybrid protein-PEG gel with or without an encapsulated growth factor. In certain embodiments, the growth factor is conjugated or unconjugated. If the growth factor is conjugated with an azide or alkyne functionality, the growth factor can be covalently incorporated within the crosslinked gel. If the growth factor is unconjugated and in its native state, it may simply be physically encapsulated within the formed gel because the SPAAC reaction is bio-orthogonal to the functional groups on the growth factors.

In another embodiment, the method further comprises increasing crosslinking of conjugated biomolecules (in the absence of any specific growth factors) on or within a tissue. Exemplary conjugated biomolecules include azide- or alkyne-conjugated collagen, fibronectin, or laminin (or combinations of these). Exemplary multi-arm PEG-NHS linkers include 3-arm PEG, 4-arm PEG, 6-arm PEG, and 8-arm PEG as well as branched dendrimers of PEG. Mixing azide- or alkyne functionalized collagen with multi-armed PEG conjugated with the counterpart azide- or alkyne-functionality), effectively creates a hybrid protein-PEG gel. In yet another embodiment, a purely biomolecule-based crosslinked gel can be formed. In this scheme, a biomolecule is prepared in two separate conjugated batches. One batch is conjugated with azide functional groups, whereas the other batch is conjugated with alkyne functional groups. Upon mixing of these conjugated biomolecules, SPAAC ensues, resulting in formation of a gel matrix comprising the crosslinked biomolecules. For example, the biomolecule used may be collagen, wherein azide-conjugated collagen and alkyne-conjugated collagen are mixed together to form a crosslinked collagen gel. In another embodiment, a conjugated growth factor or growth factor is crosslinked into the matrix by conjugation of the growth factor with either an azide or alkyne group. In another embodiment, a native (unconjugated) growth factor or growth factors are physically encapsulated within the SPAAC-crosslinked protein gel matrix.

In yet other embodiments, the gel matrices formed according to the present invention are created in combination with other therapeutic factors such as antibiotic agents, antifibrotic agents, anti-inflammatory agents, chemotherapeutic (anti-oncologic) agents, anti-angiogenic agents, or anti-thrombotic agents, or pro-thrombotic agents.

In another aspect, the invention includes a method of treating damaged tissue in a subject, the method comprising: a) contacting the damaged tissue with a mixture comprising a hydrogel-forming molecule and stem cells; and b) forming a hydrogel in situ on the damaged tissue by using a biocompatible non-photochemical bioconjugation method to crosslink the hydrogel-forming molecule, such that the hydrogel encapsulates the stem cells, wherein the encapsulated stem cells secrete growth factors that promote tissue regeneration or repair. In one example, a gel comprising azide-and alkyne-conjugated biomolecules is used to encapsulate cells such as human mesenchymal stem cells (hMSCs) or other types of stem cells or other types of differentiated cells including, but not limited to, epithelial, cartilage, bone, liver, cardiac, stromal, endothelial, nerve, corneal, retinal, muscle, and adipose cells. Such an encapsulation matrix is useful as a scaffold for tissue regeneration, or as a “living reservoir” of secreted growth factors originating from the encapsulated cells. In another example, a hybrid gel comprising alkyne (or azide)-conjugated biomolecules and a multi-arm linker such as a multi-arm PEG azide (or alkyne) is used to encapsulate cells such as human mesenchymal stem cells (hMSCs) or other types of stem cells, or other types of differentiated cells including, but not limited to, epithelial, cartilage, bone, liver, cardiac, stromal, endothelial, nerve, corneal, retinal, muscle, and adipose cells. Such an encapsulation matrix is also useful as a scaffold for tissue regeneration, or as a “living reservoir” of secreted growth factors originating from the encapsulated cells (e.g. in the case of hMSCs, for instance). In other embodiments, growth factors are either physically entrapped or chemically crosslinked (via e.g. SPAAC as described herein) alongside the encapsulated cells in order to promote or direct their growth and differentiation.

Exemplary hydrogel-forming molecules include glycoproteins, carbohydrates, and other macromolecules, including, but not limited to, various types of collagen, fibronectin, chitosan, laminin, hyaluronic acid, chondroitin sulfate, heparan sulfate, dermatan sulfate, chondroitin sulfate, and synthetic macromolecules such as polyethylene glycol (PEG), polyvinyl pyrrolidone, poly(lactic-co-glycolic acid) (PLGA), polycaprolactone, polyacrylic acid, polyvinyl alcohol, or combinations or derivatives of these. For instance, multi-arm PEG with various end-group functionalities can be used alone or in combination with other macromolecules or biomolecules. These molecules can be further engineered with degradable spacer arms such as succinate or glurarate functionalities. Branched dendrimers with various end-group functionalities may also be used. In one embodiment, the hydrogel-forming protein is collagen type I, and in another it is a hybrid of collagen type I and multi-arm (e.g. 4-arm or 8-arm) PEG.

In certain embodiments, at least one growth factor is selected from the group consisting of epidermal growth factor (EGF) and nerve growth factor (NGF). In one embodiment, both EGF and NGF are encapsulated in the hydrogel.

In another embodiment, an in situ forming hydrogel is used as a filler such as a cosmetic filler (e.g. collagen or hyaluronic acid dermal filler).

In another embodiment, the invention includes a method of treating damaged tissue in a subject, the method comprising: a) providing a mixture comprising a hydrogel-forming molecule and at least one growth factor capable of promoting tissue regeneration or repair; b) forming a growth factor-eluting hydrogel in situ over the damaged tissue by using copper-free click chemistry (e.g. strain-promoted azide-alkyne cycloaddition (SPAAC) click chemistry) to crosslink the hydrogel-forming molecule, wherein the hydrogel encapsulates the at least one growth factor.

In another embodiment, the method further comprises crosslinking at least one growth factor to the hydrogel-forming molecule within the hydrogel using SPAAC click chemistry, the method comprising: a) reacting at least one growth factor with an azide-N-hydroxysuccinimide (NHS) crosslinker to produce an azide-conjugated growth factor; b) reacting the hydrogel-forming molecule with an alkyne-NHS crosslinker to produce an alkyne-conjugated hydrogel-forming molecule; and c) reacting the azide-conjugated growth factor with the alkyne-conjugated hydrogel-forming molecule thereby crosslinking the hydrogel-forming molecule and the growth factor within the hydrogel.

In another embodiment, the method further comprises crosslinking at least one growth factor to the hydrogel-forming molecule within the hydrogel using SPAAC click chemistry, the method comprising: a) reacting the at least one growth factor with an alkyne-NHS crosslinker to produce an alkyne-conjugated growth factor; b) reacting the hydrogel-forming molecule with an azide-NHS crosslinker to produce an azide-conjugated hydrogel-forming molecule; and c) reacting the alkyne-conjugated growth factor with the azide-conjugated hydrogel-forming molecule thereby crosslinking the hydrogel-forming molecule and the at least one growth factor within the hydrogel.

In another embodiment, the method further comprises increasing crosslinking of the hydrogel using a multi-arm PEG linker comprising an azide or alkyne group. Exemplary multi-arm PEG-NHS linkers include 3-arm PEG, 4-arm PEG, 6-arm PEG, and 8-arm PEG.

In another embodiment, a hybrid co-polymer gel is produced by crosslinking an azide-conjugated first protein with an azide-conjugated second protein (or other macromolecule) using SPAAC click chemistry. For example, an azide-conjugated collagen (or other hydrogel-forming protein) can be crosslinked with an alkyne-conjugated multi-arm PEG or an alkyne-conjugated laminin or fibronectin.

In other embodiments, bioconjugation comprises performing thiol-ene click chemistry, wherein a thiol group is reacted with an alkene group via Michael addition. In one embodiment, thiol-ene click chemistry is used to produce a hydrogel by crosslinking a thiolated macromolecule with an acrylate-functionalized macromolecule. Hydrogels can be produced in this manner using suitable hydrogel-forming proteins, polymers or macromolecules, including, but not limited to, glycoproteins, carbohydrates, and other macromolecules, including, but not limited to, various types of collagen, fibronectin, chitosan, elastin, laminin, hyaluronic acid, chondroitin sulfate, heparan sulfate, dermatan sulfate, chondroitin sulfate, and synthetic macromolecules such as polyethylene glycol, polyvinyl pyrrolidone, or polyvinyl alcohol. In one embodiment, the hydrogel-forming molecule is hyaluronic acid, wherein thiolated hyaluronic acid is crosslinked with acrylate-functionalized hyaluronic acid to produce a hydrogel.

In another embodiment, in situ gel formation is accomplished using multi-functional succinimidyl esters of polyethylene glycol (PEG). Hydroxysuccinimide (NHS) ester-activated PEG linkers react efficiently with primary amino groups (—NH₂) at a pH ranging from about 7 to about 9 to form stable amide bonds. Proteins such as collagen and growth factors have multiple primary amine groups available for coupling with NHS-activated reagents. In one embodiment, a multi-functional PEG-NHS is mixed with a hydrogel-forming protein (e.g., collagen) and at least one growth factor to form a growth-factor conjugated hydrogel on the surface of a tissue.

In another example, a hybrid gel comprising a biomolecule and a multi-arm linker such as multi-arm PEG N-hydroxysuccinimide is used to encapsulate cells such as human mesenchymal stem cells (hMSCs) or other types of stem cells or other types of differentiated cells including but not limited epithelial, cartilage, bone, liver, cardiac, stromal, endothelial, nerve, corneal, retinal, muscle, and adipose cells. Such an encapsulation matrix is also useful as a scaffold for tissue regeneration, or as a “living reservoir” of secreted growth factors originating from the encapsulated cells. In other embodiments, growth factors are chemically crosslinked together with the encapsulated cells in order to promote or direct their growth and differentiation. In one embodiment, the biomolecule being crosslinked is an extracellular matrix protein such as collagen, fibronectin, or laminin, or a polypeptide like polylysine. In some embodiments, a multi-arm PEG-NHS linker is reacted with biomolecules that contain primary amines (such as the amines on lysines) to form a crosslinked structure.

In another aspect, the invention includes a method of treating damaged tissue in a subject, the method comprising: a) contacting the damaged tissue with a mixture comprising a hydrogel-forming molecule and stem cells; b) forming a hydrogel in situ on the damaged tissue by using a biocompatible non-photochemical bioconjugation method to crosslink the hydrogel-forming molecule, such that the hydrogel encapsulates the stem cells, wherein the encapsulated stem cells secrete growth factors that promote tissue regeneration or repair. Preferably, high cell viability is retained after crosslinking (i.e., greater than 80% of all stem cells are viable, preferably greater than 90-95%, and more preferably, more than 97-99% of all stem cells are viable.

Stem cells from embryos, umbilical cord, or adult tissues, or induced pluripotent stem cells may be encapsulated in the hydrogel. Such stem cells may be totipotent, multipotent, or unipotent. In one embodiment, the stem cells are human. In another embodiment, the stem cells are mesenchymal stem cells.

In certain embodiments, growth factors and stem cells are encapsulated within the same hydrogel. A growth factor may be selected in order to stimulate growth and/or differentiation of the encapsulated stem cells. The gels may further comprise a degradable spacer arm or moieties to allow their degradation in the body of a subject undergoing treatment as described herein.

In another embodiment, the invention includes a method of treating damaged tissue in a subject, the method comprising: a) contacting the damaged tissue with a mixture comprising a hydrogel-forming molecule and stem cells; b) forming a hydrogel in situ on the damaged tissue by using strain-promoted azide-alkyne cycloaddition (SPAAC) click chemistry or thiol-ene click chemistry to crosslink the hydrogel-forming molecule, such that the hydrogel encapsulates the stem cells, wherein the encapsulated stem cells secrete growth factors that promote tissue regeneration or repair. Stem cells may, for example, reduce scarring, neovascularization, or inflammation and/or stimulate epithelialization of the damaged tissue. In one embodiment, the in situ forming crosslinked matrix is used to delivery encapsulated drugs, growth factors, cells, or the secreted factors of the encapsulated cells (or combinations thereof) to an area of the body.

In certain embodiments, treatment as described herein is applied to damaged tissue at a surface or to a subsurface (e.g. via injection). For example, an in situ-forming gel may be applied at the surface of tissue (e.g., to promote wound closure) or beneath the surface (e.g. in stromal or subcutaneous tissue, such as in the subconjunctival space, submucosal space, subretinal space, suprachoroidal space, subdural space, epidural space, evacuated lens capsule, etc . . . ). In one embodiment, an in situ-forming gel is applied at the location of a damaged nerve (e.g., to promote nerve regeneration).

In another embodiment, performing SPAAC click chemistry comprises: a) reacting a first solution comprising the hydrogel-forming protein with an alkyne-NHS crosslinker to produce an alkyne-conjugated hydrogel-forming protein; b) reacting a second solution comprising the hydrogel-forming protein with an azide-NHS crosslinker to produce an azide-conjugated hydrogel-forming protein; and c) crosslinking the azide-conjugated hydrogel-forming protein with the alkyne-conjugated hydrogel-forming protein to form the hydrogel.

In another embodiment, performing thiol-ene click chemistry comprises reacting a thiolated macromolecule with an acrylate-functionalized macromolecule. For example, a thiolated hydrogel forming molecule (e.g., thiolated hyaluronic acid) can be crosslinked with an acrylate-functionalized hydrogel-forming molecule (e.g., acrylate-functionalized hyaluronic acid) to produce a hydrogel. In certain embodiments, the method further comprises crosslinking a thiolated growth factor with the acrylate-functionalized hydrogel-forming molecule, or crosslinking the thiolated hydrogel-forming molecule with an acrylate-functionalized growth factor.

Any appropriate mode of administration may be used for treating damaged tissue in a subject by the methods described herein. In certain embodiments, compositions (e.g., growth factors, hydrogel-forming proteins, stem cells, azide-NHS crosslinkers, alkyne-NHS crosslinkers) are administered topically, subcutaneously, by injection or infusion. The compositions may be administered locally to a wound or adjacent to a wound. In one embodiment, a wound dressing comprising one or more growth factors, hydrogel-forming proteins, stem cells, and/or reagents for performing SPAAC is applied to the damaged tissue. The wound dressing may comprise, for example, a gel, a viscoelastic solution, putty, a physical matrix or a membrane.

In certain embodiments, the tissue damage comprises a chemical injury, a nerve injury, a wound caused by trauma, a surgical wound, or damage caused by a disease. In certain embodiments, the tissue damage comprises damage to ocular tissue (e.g., neurotrophic keratopathy, recurrent corneal erosion, a corneal ulcer, exposure keratopathy, various other forms of retinal disease or degeneration, damage or disease to the optic nerve, or damage caused by physical trauma).

Treatment by the methods described herein (e.g., use of growth factor-loaded gels, encapsulated stem cells, and direct crosslinking of growth factors to tissue) can be applied to any tissue in need of regeneration or repair, including but not limited to skin, muscle, mucosa, nerve tissue, retinal tissue, vascular tissue, ocular tissue, bone, cartilage, and tissue in other areas of the body. Such treatment may, for example, accelerate healing of damaged tissue, increase thickness of an epithelial layer of damaged tissue, increase the rate of epithelialization at the site of damaged tissue, shorten the time required for wound closure, or stimulate nerve regeneration in damaged tissue.

In certain embodiments, multiple cycles of a treatment as described herein are administered to the subject for a time period sufficient to effect at least a partial healing of the damaged tissue or more preferably, for a time period sufficient to effect a complete healing of the damaged tissue or wound closure.

The treatment methods of the invention may be combined with any other appropriate treatment. In certain embodiments, a subject is further treated with one or more other therapeutic drugs or agents, such as, but not limited to, an antibiotic, an analgesic or anesthetic agent, an anti-inflammatory agent, a chemotherapeutic agent, an anti-metabolic agent, an anti-angiogenic agent, and an anti-hemorrhagic agent. Such therapeutic agents can be applied to the surface of damaged or abnormal tissue. Alternatively, therapeutic agents can be injected into a lesion.

In certain embodiments, one or more therapeutic agents are derivatized to allow crosslinking to damaged or abnormal tissue at the site of a lesion. For example, an alkyne-sulfo-NHS crosslinker can be injected into the tissue, such that the tissue is derivatized with alkyne groups. Next, an azide-functionalized therapeutic agent (e.g. antibiotic, chemotherapeutic agent, or other therapeutic agent) can be injected into the tissue to allow crosslinking of the agent to the tissue, such that the agent remains localized to the lesion, thereby increasing its residence time and effect. This method can be used to avoid systemic injection of an agent and to treat hard-to-reach lesions in the body that are otherwise only accessed typically by radiologic guidance (e.g. CT-guided injection), percutaneously, catheter and guidewire, laparoscopy or endoscopy.

In certain embodiments, the hydrogel-forming protein is collagen. However, other proteins, glycoproteins, carbohydrates, or other macromolecules can be used such as other types of collagen, fibronectin, chitosan, laminin, hyaluronic acid, chondroitin sulfate, heparan sulfate, dermatan sulfate, chondroitin sulfate, or synthetic macromolecules such as polyethylene glycol, polyvinyl pyrrolidone, or polyvinyl alcohol, or combinations and/or derivatives thereof.

Other types of bioconjugation chemistries can also be used. For instance, thiol-ene chemistry is a form of click chemistry where a thiol group reacts with an acrylate group via Michael addition. In this case, a thiolated macromolecule such as hyaluronic acid can form a gel upon reaction with an acrylate-functionalized hyaluronic acid (or other macromolecule).

Hybrid and co-polymer gels can also be created by mixing an azide-conjugated first protein with an azide-conjugated second protein (or other macromolecule). For instance, an azide-conjugated collagen can react with an alkyne-conjugated multi-arm PEG, or an alkyne-conjugated laminin, etc . . . Interpenetrating or semi-interpenetrating polymer networks are also possible, wherein one network is formed by SPAAC (for instance, a collagen gel formed by SPAAC as described herein) and a second network is formed by thiol-ene click chemistry such as hyaluronic acid matrices described herein. To form a semi-interpenetrating polymer network, only one of these two network is covalently crosslinked and the other is not, e.g. collagen crosslinked by SPAAC in the presence of uncrosslinked (linear) hyaluronic acid, or hyaluronic acid crosslinked by thiol-ene click chemistry in the presence of uncrosslinked collagen.

In another embodiment, in situ gel formation is accomplished with multi-functional succinimidyl esters of polyethylene glycol (PEG). Hydroxysuccinimide (NHS) ester-activated PEG linkers react efficiently with primary amino groups (—NH₂) in pH 7-9 buffers to form stable amide bonds. Proteins such as collagen and growth factors have multiple primary amines available as targets for coupling with NHS-activated reagents. The amine-reactive NHS moieties on the multi-arm PEG have the added advantage of enabling adhesion to stromal tissue. For example, active NHS esters can be used to create bonds between the amine groups in human tissue and those in a chondroitin-sulfate-NHS based adhesive. Multi-functional PEG-NHS esters also provide a systematic way to tune the mechanical and adhesive properties of the collagen gel to optimize its effects on wound healing. In one embodiment, multi-functional PEG-NHS is mixed with a protein (e.g., collagen) and a growth factor to form a growth-factor conjugated gel on a tissue surface. In another embodiment, multi-functional PEG-NHS is mixed with a protein (e.g., collagen) without a growth factor to form a growth-factor conjugated gel on a tissue surface. In yet another embodiment, multi-functional PEG-NHS is mixed with a protein (e.g., collagen) and one or more other biomolecules (e.g., hyaluronic acid) to form an interpenetrating or semi-interpenetrating polymer network of the protein and other biomolecules.

In certain embodiments, a first bioconjugation step is carried out outside the body of a subject and a second bioconjugation step is carried out in situ on living tissue.

These and other embodiments of the subject invention will readily occur to those of skill in the art in view of the disclosure herein.

### BRIEF DESCRIPTION OF THE FIGURES

FIGS. 1A-1C show schematics for the use of SPAAC click chemistry for ocular wound healing, including in situ gel formation (FIG. 1A), in situ coupling of biomolecules to the stromal surface (FIG. 1B) and in situ hMSC encapsulation (FIG. 1C). FIG. 1A shows growth factors loaded and released from an in situ formed gel crosslinked by SPAAC click chemistry at the wound bed to stimulate rapid epithelialization. FIG. 1B shows growth factors coupled directly to stromal collagen through SPAAC click chemistry to foster epithelialization and nerve regeneration. FIG. 1C shows hMSCs encapsulated within an in situ forming gel crosslinked by SPAAC click chemistry at the wound bed to treat traumatic and chemical injury to the ocular surface.

FIG. 2 shows results from Western blot analyses of various EGF-FITC binding experiments to corneal explants. Lanes shown for: (I) ladder, (II) UV-crosslinking, (III) topical only, (IV) UV-azide crosslinking, (V) SPAAC click chemistry crosslinking, and (VI) negative control (native cornea) samples.

FIG. 3A shows multi-arm succinimidyl active esters of PEG used to create protein crosslinks between collagen molecules and immobilize growth factors within collagen gels as well as to encapsulate hMSCs. FIG. 3B shows heterobifunctional crosslinking reagents containing active succinimidyl esters, including azide-PEG-NHS and dibenzocyclooctyne (DBCO)-sulfo-NHS, which can be used to conjugate growth factors to stromal collagen with SPAAC click chemistry functional groups (azide and alkyne moieties).

FIG. 4A shows formation of collagen gels using multi-arm PEG-NHS. FIG. 4B shows rheology data showing gelation of the collagen-PEG gels, as noted by the nearly 1000-fold increase in the storage modulus over 15 minutes, FIG. 4C shows results from preliminary EGF release experiments. An ELISA assay was and is being used to quantify the time-dependent release of EGF from the collagen gels, through both collagenase and hydrolytic degradation. FIG. 4D shows results from peel force adhesion experiments. The addition of 4-arm PEG as a crosslinker enables tissue-tissue adhesion that is not seen with collagen-only physical gels.

FIG. 5 shows a pilot corneal wound healing study performed in rodents, comparing EGF linked to the wound bed by SPAAC, compared to the individual components of SPAAC (DBCO-sulfo-NHS linker and azide-functionalized EGF) and saline alone. The SPAAC treatment was well-tolerated with faster wound area reduction at 24 hours in the limited number of eyes treated to date.

FIG. 6 shows a Western blot to detect applied NGF-FITC within corneal stroma (left to right: ladder, untreated cornea, NGF-FITC control, topically applied NGF-FITC, and SPAAC-crosslinked NGF-FITC.

FIGS. 7A and 7B show live-dead assays. Greater than 97% cell viability was observed 72 hours after (FIG. 7A) direct keratocyte exposure to NHS-conjugation followed by SPAAC click chemistry and (FIG. 7B) NHS-crosslinking of collagen to encapsulate hMSCs.

FIGS. 8A and 8B show that SPAAC click chemistry can be used to rapidly form crosslinks in several different ways: (FIG. 8A) between collagen fibrils and (FIG. 8B) between growth factors and collagen fibrils. Exogenous collagen is crosslinked to itself, to exogenous growth factors, and to corneal collagen to form a growth-factor-loaded, adherent collagen gel to cover wounds. Exogenous growth factors are bound directly to tissue collagen. Exogenous collagen encapsulates hMSCs onto the surface of wounds by binding to other exogenous collagen molecules as well as to collagen in the wounded tissue.

FIG. 9A shows rheology data showing gelation of collagen gels with varying length spacer arms. FIG. 9B show ELISA data from EGF release experiments from collagen gels, with or without collagenase exposure and with or without chemical linkage of EGF-azide into the gel via SPAAC. FIG. 9C shows a tissue section of corneal stroma with fluorescein isothiocyanate (FITC)-labeled collagen gel formed and covalently bound to corneal stroma via SPAAC.

FIG. 10A shows real-time surface plasmon resonance (SPR) data showing (i) collagen coating on gold, followed by (ii) alkyne-conjugation of collagen, (iii) blocking with ethanolamine, and then (iv) click chemistry reaction with azide-functionalized NGF. FIG. 10B shows a more granular view of SPR data showing increased binding of NGF to collagen-coated gold surfaces compared to physical adsorption of NGF.

FIG. 11 shows ELISA quantification of surface concentration of EGF as a function of reaction time using SPAAC.

FIG. 12 shows NGF-FITC binding experiments showing (1) SPAAC for 10 minutes, (2) SPAAC for 30 minutes, (3) topical delivery for 10 minutes, (4) topical delivery for 1 minute, and (5) no treatment.

FIG. 13A shows cell seeding on EGF-bound collagen surfaces yielded greater proliferation of senescent primary CECs over 5 days compared to surfaces without chemically bound EGF. FIG. 13B shows cell proliferation assay with hMSCs encapsulated within a collagen in a permeable transwell insert over primary CECs. The secreted factors produced by the encapsulated hMSCs dramatically increased the growth of CECs.

FIG. 14 shows binding assay data for biotinylated EGF binding to the EGF receptor. Binding of EGF to its receptor by a competitive (non-biotinylated) EGF ligand reduces the relative intensity of the signal in this assay. The results showed that while heat-denatured EGF did not interfere with biotinylated EGF-EGFR binding, both non-denatured (native) EGF and azide-conjugated EGF do (without a statistical difference between the two), demonstrating that the process of conjugating EGF with azide groups via N-hydroxysuccinimide coupling via primary amines on EGF retains the bioactivity of EGF.

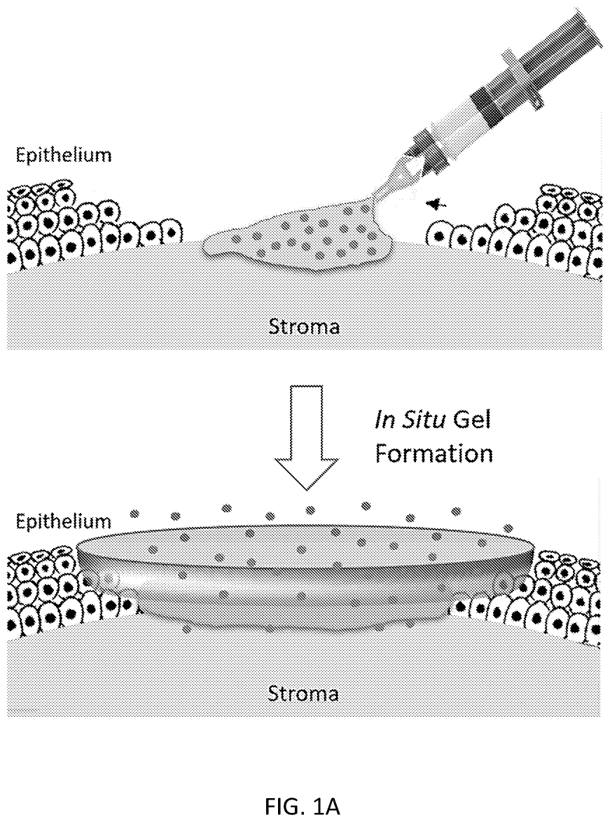

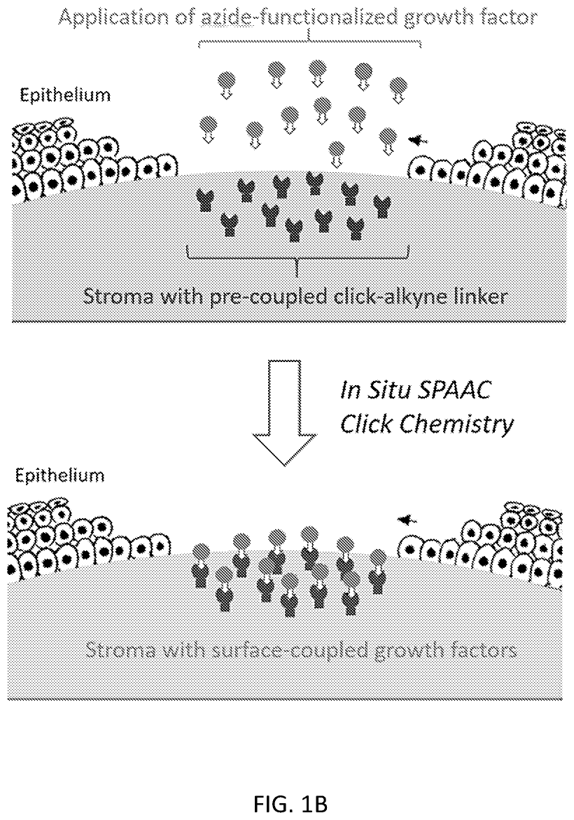

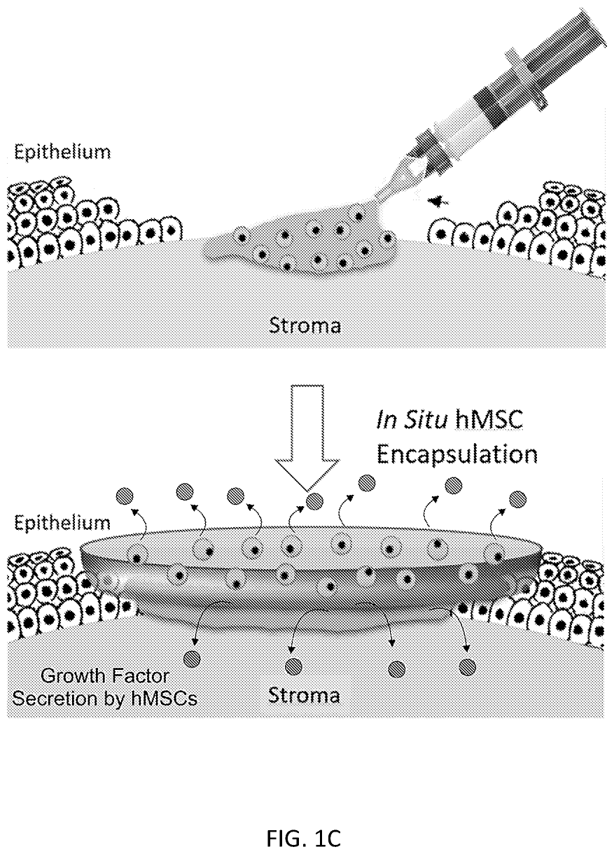

### DETAILED DESCRIPTION

The practice of the present invention will employ, unless otherwise indicated, conventional methods of medicine, pharmacology, chemistry, biochemistry, molecular biology and recombinant DNA techniques, within the skill of the art. Such techniques are explained fully in the literature. See, e.g. S. S. Wong and D. M. Jameson Chemistry of Protein and Nucleic Acid Cross-Linking and Conjugation (CRC Press, 2^{nd} edition, 2011); G. T. Hermanson Bioconjugate Techniques (Academic Press, 3^{rd} edition, 2013); B. Bowling Kanski's Clinical Ophthalmology: A Systematic Approach, 8e (Saunders Ltd., 8^{th} edition, 2015); A. L. Lehninger, Biochemistry (Worth Publishers, Inc., current addition); Sambrook et al., Molecular Cloning: A Laboratory Manual (3^{rd} Edition, 2001); and Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a biomolecule” includes two or more biomolecules, and the like.

The term “about,” particularly in reference to a given quantity, is meant to encompass deviations of plus or minus five percent.

A “wound” is a break or discontinuity in the structure of an organ or tissue, including epithelium, connective tissue, and muscle tissue. Examples of wounds include, but are not limited to, skin wounds, burns, bruises, ulcers, bedsores, grazes, tears, cuts, punctures, perforations, corneal abrasions and disruptions, corneal damage caused by neurotrophic keratopathy and exposure keratopathy, and neurotrophic recurrent corneal erosions. A wound may include tissue damage produced by a surgical procedure, trauma, or disease.

“Topical” application refers to non-systemic local administration of an active ingredient (e.g., biomolecule or photosensitizer) to a surface or subsurface of damaged tissue or a wound.

The term “subject” includes both vertebrates and invertebrates, including, without limitation, mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like.

“Treatment” of a subject or “treating” a subject for a disease or condition herein means reducing or alleviating clinical symptoms of the disease or condition, including tissue damage or loss, nerve damage, or impaired or slow wound-healing.

By “therapeutically effective dose or amount” of a biomolecule or stem cells is intended an amount that, when administered as described herein, brings about a positive therapeutic response in a subject having tissue damage or loss, such as an amount that improves wound healing or nerve regeneration. A therapeutically effective amount of a biomolecule or stem cells may, for example, accelerate healing of damaged tissue, increase thickness of an epithelial layer of the damaged tissue, increase rate of epithelialization at the site of damaged tissue, shorten the time required for wound closure, or promote nerve regeneration in the damaged tissue. The exact amount required will vary from subject to subject, depending on the species, age, and general condition of the subject, the severity of the condition being treated, mode of administration, and the like. Additionally, an “effective amount” of a reagent for performing SPAAC click chemistry (e.g., heterobifunctional crosslinking agents for attaching suitable azide and alkyne moieties to molecules) is an amount sufficient for crosslinking biomolecules or hydrogel-forming proteins in situ on tissue. An appropriate “effective” amount in any individual case may be determined by one of ordinary skill in the art using routine experimentation, based upon the information provided herein.

The terms “peptide,” “oligopeptide,” and “polypeptide” refer to any compound comprising naturally occurring or synthetic amino acid polymers or amino acid-like molecules including but not limited to compounds comprising amino and/or imino molecules. No particular size is implied by use of the terms “peptide,” “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Preferably, the polypeptide is between about 40 and 300 residues in length. Generally, one skilled in art can easily select the maximum length in view of the teachings herein. Further, peptides and polypeptides, as described herein, for example synthetic peptides, may include additional molecules such as labels or other chemical moieties. Such moieties may further enhance stimulation of epithelial cell proliferation and/or wound healing, and/or nerve regeneration, and/or biomolecule stability or delivery.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, glycosylation, acetylation, phosphorylation, and the like.

Amino acid derivatives can also include modifications to the native sequence, such as deletions, additions and substitutions (generally conservative in nature), so long as the polypeptide or peptide maintains the desired activity (e.g., promote epitheilial cell proliferation and wound healing). These modifications may be deliberate, as through site-directed mutagenesis, or may be accidental, such as through mutations of hosts that produce the proteins or errors due to PCR amplification. Furthermore, modifications may be made that have one or more of the following effects: increasing specificity or efficacy of biomolecule, enhancing epithelial cell proliferation, wound healing, and/or nerve regeneration, and facilitating cell processing.

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

“Biocompatible” refers to a material that is non-toxic to a cell or tissue.

As used herein, the term “cell viability” refers to a measure of the number of cells that are living or dead, based on a total cell sample. High cell viability, as defined herein, refers to a cell population in which greater than 80% of all cells are viable, preferably greater than 90-95%, and more preferably a population characterized by high cell viability containing more than 97-99% viable cells.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention is based on the discovery that in situ bioconjugation can be used to deliver therapeutic biomolecules and stem cells to enhance wound healing. The inventors have used SPAAC click chemistry in a variety of ways, including for production of an in situ-forming, growth factor-eluting membrane that covers wounds, direct covalent linkage of growth factors to damaged tissue, and encapsulation of stem cells in a biocompatible carrier matrix at the surface of damaged tissue (see Examples). In particular, the inventors have applied their methods with EGF (e.g., stimulates rapid re-epithelialization), NGF (e.g., stimulates nerve regeneration), and encapsulated human mesenchymal stem cells (e.g., to reduce scarring, neovascularization, and inflammation while promoting epithelialization) to enhance healing of damaged ocular tissue.

In order to further an understanding of the invention, a more detailed discussion is provided below regarding methods of using bioconjugation for delivery of therapeutic biomolecules and stem cells to promote wound healing.

### A. Bioconjugation for Delivery of Therapeutic Biomolecules

Biomolecules that can be used in the practice of the invention include any biomolecule, which when administered using a biocompatible non-photochemical bioconjugation method as described herein, promotes tissue repair or regeneration. Biomolecules may, for example, accelerate healing, increase thickness of an epithelial layer, increase the rate of epithelialization, shorten the time required for wound closure, or promote nerve regeneration in damaged tissue. In certain types of wounds, one or more of the following may be used for healing: a scaffold for cell adhesion (e.g., a functional extracellular matrix), a stimulus for cell proliferation (e.g., growth factors), nerve signaling (e.g., neuropeptides), and axon guidance proteins for nerve regeneration. Exemplary biomolecules that can be used include growth factors, such as epidermal growth factor (EGF), nerve growth factor (NGF), vascular endothelial growth factor (VEGF), and insulin-like growth factor (IGF); neuropeptides, such as substance P (SP) and calcitonin gene-related peptide; extracellular matrix proteins, such as fibronectin, collagen, laminin, and fibrin; axon guidance proteins, such as netrins (e.g., netrin-1), ephrins, and cell adhesion molecules; and other biomolecules that play various roles in tissue regeneration, such as beta-thymosins (e.g., thymosin beta-4). Other types of molecules or biomolecules may also be used, such as anti-vascular endothelial growth factor (anti-VEGF) therapeutic agents to prevent vascularization, leakage, or growth. Tethering anti-VEGF therapeutic agents (e.g., bevacizumab and ranibizumab) to tissues may be useful, for example, in the treatment of certain cancers or proliferative conditions, including wet macular degeneration or diabetic retinopathy.

In one embodiment, the bioconjugation method used for crosslinking is SPAAC, a Cu-free variation of click chemistry that is generally biocompatible with cells. SPAAC utilizes a substituted cyclooctyne having an internal alkyne in a strained ring system. Ring strain together with electron-withdrawing substituents in the cyclooctyne promote a [3+2] dipolar cycloaddition with an azide functional group. SPAAC can be used for bioconjugation and crosslinking by attaching azide and cyclooctyne moieties to molecules. For a description of SPAAC, see, e.g., Baskin et al. (2007) Proc Natl Acad Sci USA 104(43):16793-16797, Agard et al. (2006) ACS Chem. Biol. 1: 644-648, Codelli et al. (2008) J. Am. Chem. Soc. 130:11486-11493, Gordon et al. (2012) J. Am. Chem. Soc. 134:9199-9208, Jiang et al. (2015) Soft Matter 11(30):6029-6036, Jang et al. (2012) Bioconjug Chem. 23(11):2256-2261, Ornelas et al. (2010) J Am Chem Soc. 132(11):3923-3931; herein incorporated by reference in their entireties.

Heterobifunctional crosslinking agents can be used to attach suitable azide and alkyne moieties to molecules for performing SPAAC. In particular, reactions with N-hydroxysuccinimide (NHS) can be used for bioconjugation of proteins such as collagen, elastin, and growth factors, which have multiple primary amines available as targets for coupling with NHS-activated reagents. Exemplary alkyne-NHS-crosslinker agents include dibenzycyclooctyne-N-hydroxysuccinimide (DBCO-NHS), bicyclononyne-N-hydroxysuccinimide (BCN-NHS), and dibenzocyclooctyne-sulfo-N-hydroxysuccinimide (DBCO-sulfo-NHS). Exemplary azide-NHS crosslinker agents include azide-polyethylene glycol (PEG)-NHS crosslinkers with PEG polymers of various lengths (azide-PEGₙ-NHS). The length of the PEG polymer can be used to control the spacing between the NHS and azide moieties. The PEG spacer arms may range from one PEG unit to many PEG units in length up to an average molecular weight of about 14,000 Da. The spacer arm may also comprise other types of chemical backbones, such as an aliphatic backbone. Heterobifunctional crosslinking agents suitable for performing SPAAC are commercially available from a number of companies, including JenKem Technology USA (Plano, Tex.), Sigma-Aldrich, Inc. (St. Louis, Mo.), BroadPharm (San Diego, Calif.), Quanta BioDesign (Plain City, Ohio), Thermo Fisher Scientific Inc. (Waltham, Mass.), and Nanocs Inc. (New York, N.Y.); herein incorporated by reference.

SPAAC can be used for direct covalent linkage of biomolecules to damaged tissue as well as crosslinking biomolecules with one another. Biomolecules may include more than one functional group that can be crosslinked to allow formation of bonds among multiple biomolecules and a tissue surface or subsurface. In order to crosslink biomolecules to tissue using SPAAC, the damaged tissue may be reacted with an azide-N-hydroxysuccinimide (NHS) crosslinker to produce azide-derivatized tissue, wherein proteins such as collagen in the damaged tissue are covalently coupled to azide functional groups. One or more growth factors can be reacted with an alkyne-NHS crosslinker to produce alkyne-conjugated growth factors, which can be subsequently reacted with the azide-derivatized tissue using SPAAC to covalently link the growth factors to the damaged tissue.

Alternatively, the damaged tissue can be reacted with an alkyne-NHS crosslinker to produce alkyne-derivatized tissue, wherein proteins such as collagen are covalently coupled to alkyne functional groups. One or more growth factors can be reacted with an azide-PEG-NHS crosslinker to produce azide-conjugated growth factors, which can be subsequently reacted with the alkyne-derivatized tissue using SPAAC to covalently link the growth factors to the damaged tissue.

In another embodiment, the bioconjugation method used for crosslinking is thiol-ene click chemistry. Bioconjugation using thiol-ene click chemistry involves reacting a thiol group with an alkene group via Michael addition. The thiol-ene click reaction can be optionally augmented by light (i.e., photo-click reaction). For a description of the use of thiol-ene click chemistry for crosslinking and forming hydrogels, see, e.g., Grim et al. (2015) J. Control Release 219:95-106; Scanlan et al. (2014) Molecules 19(11):19137-151; Hoyle et al. (2010) Angew Chem. Int. Ed. Engl. 49(9):1540-1573; van Dijk et al. (2009) Bioconjug Chem. 20(11):2001-2016; herein incorporated by reference in their entireties.

In certain embodiments, more than one bioconjugation step is performed. For example, at least one bioconjugation step may be performed in vitro, and at least one bioconjugation step may be performed directly on the damaged tissue. Alternatively or additionally, more than one bioconjugation technique may be used for crosslinking. For example, SPAAC click chemistry can be combined with thiol-ene click chemistry to crosslink different biomolecules. As will be clear to one of skill in the art, some bioconjugation chemistries are more advantageous depending on the conditions, tissue being treated, and the particular biomolecules, polymers, or other factors being crosslinked.

Biomolecules, e.g., suitably conjugated for SPAAC or thiol-ene click chemistry, may be applied to damaged tissue at a surface or a subsurface. For example, one or more biomolecules may be applied at the surface of tissue (e.g., to promote wound closure) or beneath the surface (e.g. in stromal or subcutaneous tissue), or at the location of a damaged nerve (e.g., to promote nerve regeneration). In certain embodiments, biomolecules are applied to damaged tissue in a pattern, tracks, or a gradient. For example, a gradient of growth factors or axon guidance factors can be used, e.g., to guide cell migration or nerve regeneration. A gradient can be produced, for example, by varying the concentration of biomolecules along damaged tissue. A particular embodiment of a gradient is in the example of injection; injecting an alkyne-containing heterobifunctional linker in to a tissue leads to diffusion from a point-source and creates a radial gradient, with highest concentration of reacted alkyne at the center and the lowest at the periphery. Subsequent injection of the azide-conjugated therapeutic agent leads to coupling of the therapeutic agent along the gradient, creating a concentration gradient of the therapeutic agent. (As discussed herein, the reverse chemistry is also possible, i.e. azide-containing heterobifunctional linkers that are injected first and attaching to tissue in a radial gradient, followed by injection of the alkyne-functionalized therapeutic. This would be especially useful for the localized delivery of antibiotic agents, antifibrotic agents, anti-inflammatory agents, chemotherapeutic (anti-oncologic) agents (for instance to a solid tumor), antibodies, anti-angiogenic agents, or anti-thrombotic agents, or pro-thrombotic agents.

In addition, damaged tissue may be prepared prior to treatment by exfoliation or debridement of fibrotic or necrotic areas.

In certain embodiments, one or more growth factors are applied to the damaged tissue. Exemplary growth factors that can be used in the practice of the invention include epidermal growth factor (EGF), nerve growth factor (NGF), vascular endothelial growth factor (VEGF), and insulin-like growth factor (IGF). In another embodiment, at least two growth factors are crosslinked to the damaged tissue. For example, EGF in combination with NGF can be crosslinked to the damaged ocular tissue, wherein EGF stimulates re-epithelialization and NGF stimulates nerve regeneration in the damaged ocular tissue. In other embodiments, viral vectors and/or genetic material (viral DNA, aptamers, RNA and their derivatives) are encapsulated within the biomolecular/macromolecular matrix gels formed by copper-free click chemistry or succinimidyl (multi-arm PEG) linkers as described in the present invention, to provide a depot or reservoir of material used for gene therapy.

In another embodiment, SPAAC or thiol-ene click chemistry is used for production of an in situ-forming, growth factor-eluting hydrogel that covers wounds. Exemplary hydrogel-forming molecules include glycoproteins, carbohydrates, and other macromolecules, including, but not limited to, various types of collagen, fibronectin, chitosan, laminin, hyaluronic acid, chondroitin sulfate, heparan sulfate, dermatan sulfate, chondroitin sulfate, and synthetic macromolecules such as polyethylene glycol, polyvinyl pyrrolidone, or polyvinyl alcohol. In particular, collagen I is useful for producing crosslinked protein hydrogels. A number of engineered elastin-like proteins have also been described for use in producing protein hydrogels (see, e.g., Straley et al. (2009) Soft Matter 5 (1):114-124, Madl et al. (2016) Adv Funct Mater 26(21):3612-3620; herein incorporated by reference).

SPAAC click chemistry can be performed with such hydrogel-forming molecules in situ to encapsulate growth factors in hydrogels over damaged tissue. For example, an alkyne-conjugated hydrogel-forming molecule can be produced by reacting the hydrogel-forming molecule (e.g., in a first solution) with an alkyne-NHS crosslinker. An azide-conjugated hydrogel-forming molecule can be produced by reacting the hydrogel-forming molecule (e.g., in a second solution) with an azide-NHS crosslinker. SPAAC is then performed to crosslink the azide-conjugated hydrogel-forming molecule with the alkyne-conjugated hydrogel-forming molecule (e.g., by mixing the first and second solutions) to form the hydrogel. A multi-arm PEG linker comprising an azide or alkyne group may be used to further increase crosslinking of the hydrogel. Exemplary multi-arm PEG linkers include 3-arm PEG, 4-arm PEG, 6-arm PEG, and 8-arm PEG. Such crosslinkers are commercially available from JenKem Technology USA (Plano, Tex.).

In addition, growth factors can be crosslinked to the hydrogel-forming molecule within the hydrogel using SPAAC. For example, growth factors can be conjugated for SPAAC with an azide-N-hydroxysuccinimide (NHS) crosslinker to produce an azide-conjugated growth factor. The hydrogel-forming molecule can be conjugated with an alkyne-NHS crosslinker to produce an alkyne-conjugated hydrogel-forming protein, which is subsequently reacted with the azide-conjugated growth factor, thereby crosslinking the hydrogel-forming molecule and the growth factor within the hydrogel. Alternatively, a growth factor can be reacted with an alkyne-NHS crosslinker to produce an alkyne-conjugated growth factor. A hydrogel-forming molecule can be reacted with an azide-NHS crosslinker to produce an azide-conjugated hydrogel-forming molecule, which is subsequently reacted with the alkyne-conjugated growth factor thereby crosslinking the hydrogel-forming molecule and the growth factor within the hydrogel.

In another embodiment, thiol-ene click chemistry is used to produce a hydrogel by crosslinking a thiolated macromolecule with an acrylate-functionalized macromolecule. Hydrogels can be produced in this manner using suitable hydrogel-forming proteins, polymers or macromolecules, such as described above. For example, thiolated hyaluronic acid can be crosslinked with acrylate-functionalized hyaluronic acid to produce a hydrogel.

In another embodiment, in situ gel formation is accomplished using multi-functional succinimidyl esters of polyethylene glycol (PEG). Hydroxysuccinimide (NHS) ester-activated PEG crosslinkers react efficiently with primary amino groups (—NH₂) at a pH ranging from about 7 to about 9 to form stable amide bonds. In particular, proteins such as collagen and growth factors have multiple primary amine groups available for coupling with NHS-activated reagents. Sulfonated crosslinkers have the advantage that they tend to be water soluble and can be applied to tissue in situ safely without an organic solvent. Amine-reactive NHS moieties on multi-arm PEG crosslinkers have the added advantage of enabling adhesion to stromal tissue. Multi-functional PEG-NHS esters also provide a systematic way to tune the mechanical and adhesive properties of a hydrogel to optimize its effects on wound healing. In one embodiment, a multi-functional PEG-NHS is mixed with a hydrogel-forming protein (e.g., collagen) and at least one growth factor to form a growth-factor conjugated hydrogel on the surface of a tissue. Exemplary multi-arm PEG-NHS linkers that can be used in the practice of the invention include 3-arm PEG, 4-arm PEG, 6-arm PEG, and 8-arm PEG.

Additionally, in situ bioconjugation, as described herein, can be used to encapsulate stem cells in hydrogels over damaged tissue to promote wound healing. Stem cells from embryos, umbilical cord, or adult tissues, or induced pluripotent stem cells may be used for this purpose. Such stem cells may be totipotent, multipotent, or unipotent. Preferably, high cell viability is retained after crosslinking (i.e., greater than 80% of all stem cells are viable, preferably greater than 90-95%, and more preferably, more than 97-99% of all stem cells are viable), and the encapsulated stem cells are capable of secreting growth factors that promote tissue regeneration or repair. Stem cells may, for example, reduce scarring, neovascularization, or inflammation and/or stimulate epithelialization of the damaged tissue. In one embodiment, the stem cells are mesenchymal stem cells from bone marrow.

For example, stem cells, mixed with a hydrogel-forming molecule, can be applied to the surface of a wound and reacted in situ with azide-NHS and alkyne-NHS crosslinkers. SPAAC click chemistry is performed in situ, crosslinking the azide-conjugated hydrogel-forming protein with the alkyne-conjugated hydrogel-forming protein, to produce a hydrogel encapsulating the stem cells at the wound site. Alternatively, in situ thiol-ene click chemistry can be performed with a thiolated hydrogel forming molecule (e.g., thiolated hyaluronic acid) and an acrylate-functionalized hydrogel-forming molecule (e.g., acrylate-functionalized hyaluronic acid) to produce a hydrogel encapsulating stem cells.

In certain embodiments, growth factors and stem cells are encapsulated within the same hydrogel. A growth factor may be selected in order to stimulate growth and/or differentiation of the encapsulated stem cells. The gels may further comprise a degradable spacer arm or moieties to allow their degradation in the body of a subject undergoing treatment as described herein.

### B. Administration

At least one therapeutically effective cycle of treatment by any of the methods described herein will be administered to a subject in need of tissue regeneration or repair. By “therapeutically effective dose or amount” of a biomolecule or stem cells is intended an amount that, when administered as described herein, brings about a positive therapeutic response in a subject having tissue damage or loss, such as an amount that improves wound healing or nerve regeneration. A therapeutically effective amount of a biomolecule or stem cells may, for example, accelerate healing of damaged tissue, increase thickness of an epithelial layer of the damaged tissue, increase rate of epithelialization at the site of damaged tissue, shorten the time required for wound closure, or promote nerve regeneration in the damaged tissue. Additionally, an “effective amount” of a reagent for performing SPAAC click chemistry or thiol-ene click chemistry (e.g., heterobifunctional crosslinking agents for attaching suitable azide, alkyne, acrylate, or thiol moieties to molecules) is an amount sufficient for crosslinking biomolecules or hydrogel-forming proteins in situ on tissue.

In certain embodiments, multiple therapeutically effective doses of compositions comprising one or more biomolecules, and/or stem cells, and/or hydrogel-forming proteins, and/or heterobifunctional crosslinking agents, and/or one or more other therapeutic agents, such as other drugs or agents for treating a wound or damaged tissue, or other medications will be administered. The compositions of the present invention are typically, although not necessarily, administered topically, via injection (subcutaneously or intramuscularly), by infusion, or locally. Additional modes of administration are also contemplated, such as transdermal, intradermal, and so forth.

The preparations according to the invention are also suitable for local treatment. Compositions comprising one or more biomolecules, stem cells, hydrogel-forming molecules, and/or heterobifunctional crosslinking agents may be administered directly on the surface of a wound, adjacent to a wound, or beneath the surface of a wound (e.g. in stromal or subcutaneous tissue). Additionally, compositions may be applied at the location of a damaged nerve (e.g., to promote nerve regeneration). For example, a composition may be administered by spraying the composition on a wound, or as drops or a topical paste. Biomolecules, stem cells, hydrogel-forming molecules, and/or heterobifunctional crosslinking agents may also be added to wound dressings. A wound dressing may comprise, for example, a gel, a viscoelastic solution, putty, a physical matrix or a membrane. The particular preparation and appropriate method of administration are chosen to deliver biomolecules and/or stem cells and effect crosslinking reactions at the site in need of tissue regeneration or repair.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising biomolecules, stem cells, hydrogel-forming molecules, heterobifunctional crosslinking agents, or other agents may be administered using the same or different modes of administration in accordance with any medically acceptable method known in the art.

In another embodiment, the pharmaceutical compositions comprising biomolecules, stem cells, hydrogel-forming molecules, heterobifunctional crosslinking agents, or other agents are administered prophylactically. Such prophylactic uses will be of particular value for subjects who suffer from a condition which impairs or slows down the healing of a wound or causes tissue damage or prior to a procedure that will cause tissue damage.

In another embodiment of the invention, the pharmaceutical compositions comprising biomolecules, stem cells, hydrogel-forming proteins, heterobifunctional crosslinking agents, or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising biomolecules (e.g. biomolecule-azide or -alkyne conjugate) or hydrogel-forming molecules (e.g. hydrogel-forming protein-azide or -alkyne conjugate, thiolated hyaluronic acid or acrylate-functionalized hyaluronic acid conjugate) as provided herein to a patient suffering from a condition that is responsive to treatment with biomolecules contained in the conjugate or composition. The method comprises administering, via any of the herein described modes, a therapeutically effective amount of the conjugate or drug delivery system, preferably provided as part of a pharmaceutical composition.

The actual dose of biomolecules, drug, or stem cells to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. The amount of biomolecules or stem cells administered will depend on the potency of particular biomolecules or stem cells and the magnitude of their effect on tissue regeneration and repair (e.g., wound epithelialization and healing, nerve regeneration) and the route of administration.

Biomolecules, stem cells, hydrogel-forming molecules, and/or heterobifunctional crosslinking agents, prepared as described herein (again, preferably provided as part of a pharmaceutical preparation), can be administered alone or in combination with one or more other therapeutic agents for treating a wound or tissue damage, such as, but not limited to, analgesic agents, anesthetic agents, antibiotics, anti-inflammatory agents, or other agents that promote wound healing, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day. In some cases, only a single administration will be needed.

### C. Applications

The methods of the invention can be applied to any number of medical applications where tissue regeneration or improved wound healing is needed. Any condition where healing is impaired may especially benefit from such treatment. For example, tissue damage caused by physical trauma, burns, chemical exposure, disease, or surgery, including, but not limited to, chemical injuries, skin injuries, nerve injuries, or eye injuries may benefit from treatment as described herein.

For example, corneal damage, particularly persistent corneal epithelial defects can be treated by using SPAAC or thiol-ene click chemistry to deliver biomolecules and/or stem cells directly to damaged stroma. In particular, damage to corneal tissue, such as caused by neurotrophic keratopathy, recurrent corneal erosion, a corneal ulcer, exposure keratopathy, or physical trauma may be treated in this manner. The corneal surface can be prepared by optionally debriding the edges of an epithelial defect and its base, followed by performing SPAAC or thiol-ene click chemistry with one or more biomolecules, stem cells, hydrogel-forming molecules, or heterobifunctional crosslinking agents, applied to the surface using sterile week-cells. An optional bandage contact lens can also be placed after the reaction.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### General Overview of Methods for Crosslinking and Tethering Biomolecules to Damaged Ocular Tissue to Promote Healing

We show that ocular wound healing is enhanced by delivering and crosslinking therapeutic factors directly to damaged tissue, which can be accomplished in three ways: (1) through an injectable, in situ-forming growth factor-eluting membrane that covers ulcers and stimulates rapid re-epithelialization, (2) by binding growth factors directly to the stromal wound bed, and (3) by encapsulating human mesenchymal stem cells (hMSCs) within a gel at the ocular surface. These approaches utilize a biocompatible form of click chemistry, known as strain-promoted azide-alkyne cycloaddition (SPAAC), as an in situ therapeutic delivery modality for biomolecules and stem cells to enhance wound healing in the eye. SPAAC can be used, for example, to bio-orthogonally encapsulate hMSCs within engineered elastin-like peptide (ELP) hydrogels (Madl et al. (2016) Advanced Functional Materials 26(21):3612-3620) and for bioconjugation of topically applied growth factors (Pastor et al. (1992) Cornea 11(4):311-314; Kawamoto et al. (2004) Prog. Brain Res. 146:369-384; Aloe et al. (2012) J. Transl. Med. 10:239-5876-10-239; Nishida et al. (1996) J. Cell Physiol. 169(1):159-166; Nakamura et al. (1997) Exp. Eye Res. 65(3):321-329) and injected hMSC suspensions (Oh et al. (2012) Molecular Therapy 20(11):2143-2152; Lan et al. (2012) Invest. Ophthalmol. Vis. Sci. 53(7):3638-3644; Ye et al. (2006) Eye 20(4):482-490; Watson et al. (2010) Br. J. Ophthalmol. 94(8):1067-1073; Omoto et al. (2009) Invest. Ophthalmol. Vis. Sci. 50(5):2109-2115) to promote ocular wound healing.

### EXAMPLE 2

### Application of SPAAC Click Chemistry to Ocular Wound Healing

Strain-promoted azide-alkyne cycloaddition (SPAAC) click chemistry, a biocompatible protein conjugation chemistry, can be safely used on living cells (Madl et al. (2016) Advanced Functional Materials 26(21):3612-3620; Baskin et al. (2007) Proc. Natl. Acad. Sci. USA 104(43):16793-16797; Chang et al. (2010) Proc. Natl. Acad. Sci. USA 107(5):1821-1826) because it does not require harmful external triggers such as UV light or a metal-ion catalyst such as copper (Takahashi et al. (2013) Biomacromolecules 14(10):3581-3588). SPAAC facilitates protein gel formation in a matter of seconds while retaining 97% or greater viability of multiple cell types, including neuronal, endothelial, and human mesenchymal stem cells (hMSCs) (Madl et al., supra).

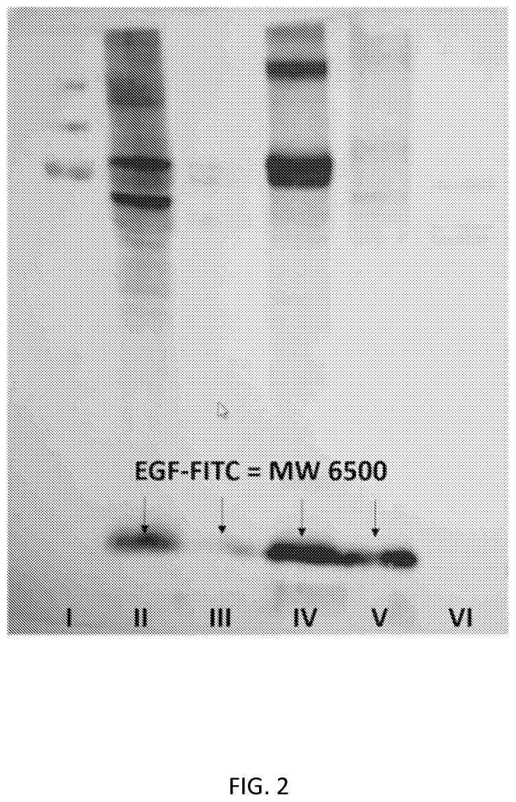

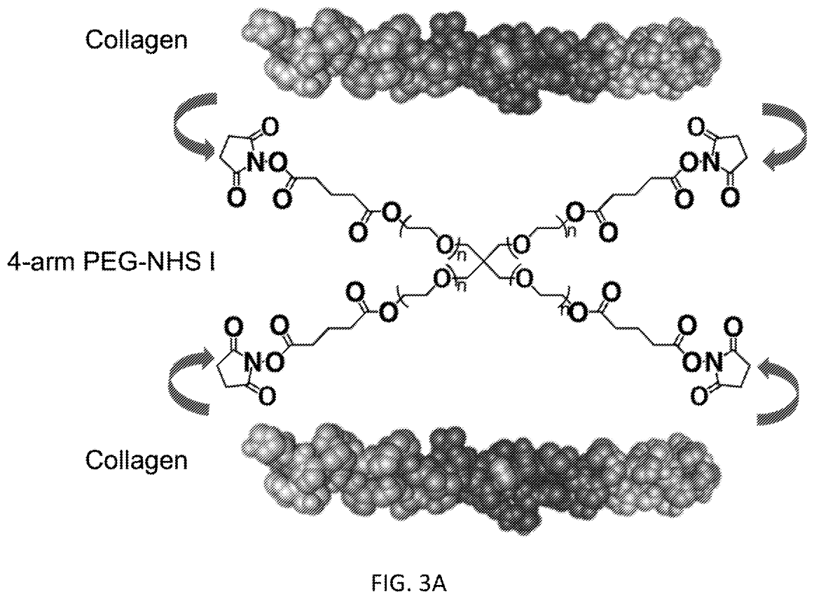

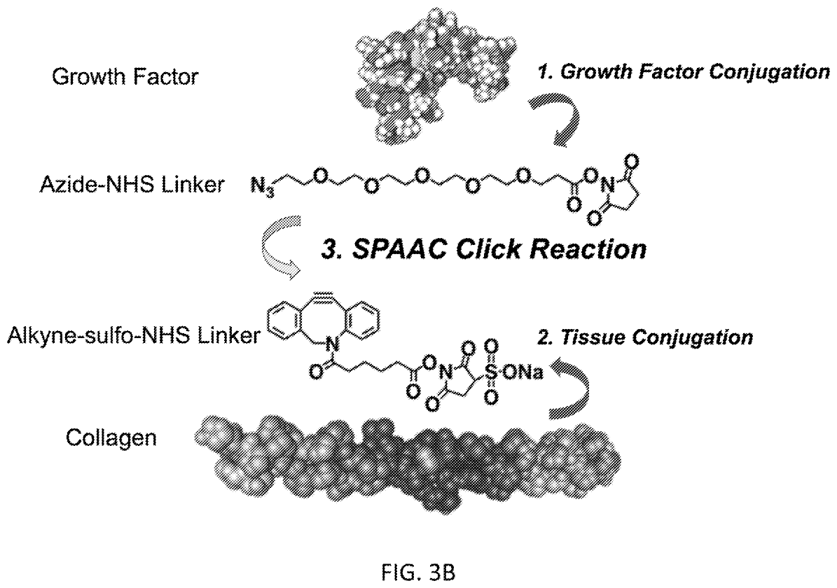

Clinically, treatment of the eye using in situ SPAAC is performed with a two-step process—with the initial step being topical application of a heterobifunctional, polyethylene glycol (PEG)-based azide-N-hydroxysuccinimide (NHS) linker that rapidly couples azide groups to stromal collagen, followed by exposure of the modified wound bed to alkyne-conjugated growth factors leading to SPAAC (FIG. 2). PEG-NHS-ester chemistry is also highly biocompatible and has long been used as the basis of FDA-approved surgical sealants (Kim et al. (2011) Spine (Phila Pa. 1976) 36(23):1906-1912), including the recently cleared ReSure sealant (Ocular Therapeutix) (Matossian et al. (2015) Clin. Ophthalmol. 9:921-928; Masket et al. (2014) Journal of Cataract & Refractive Surgery 40(12):2057-2066). NHS ester-activated PEG linkers react efficiently with primary amino groups (—NH₂) in pH 7-9 buffers to form stable amide bonds. Proteins such as collagen and growth factors have multiple primary amines available as targets for coupling with NHS-activated reagents. In situ NHS chemistry yields stable amide bonds that serve as “tissue anchors” upon which the SPAAC-crosslinked gels and growth factors can be bonded to stromal tissue. The amine-reactive NHS moieties on multi-arm PEG linkers have the added advantage of enabling adhesion to stromal tissue (Sargeant et al. (2012) Acta Biomaterialia 8(1):124-132).

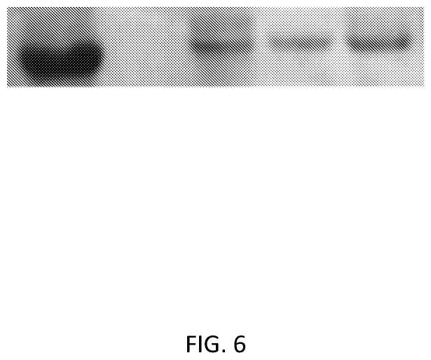

For example, to achieve direct growth factor attachment to the wound bed, NHS chemistry can be used to conjugate stromal collagen with alkyne functional groups and growth factors with azide groups that can subsequently react with each other through SPAAC click chemistry (FIGS. 6A and 6B). A water-soluble, heterobifunctional alkyne-NHS linker can be applied topically with a Weck-Cell to couple the alkyne group to tissue collagen, followed by exposure of the stromal bed to an azide-conjugated growth factor, also applied with a Weck-Cell. SPAAC and surface conjugation occur within minutes with no further side reactions.

### EXAMPLE 3

### Collagen Gels Formed in Situ

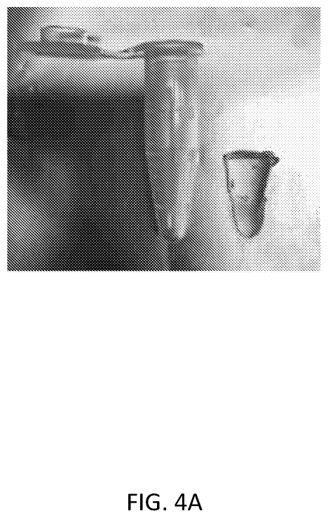

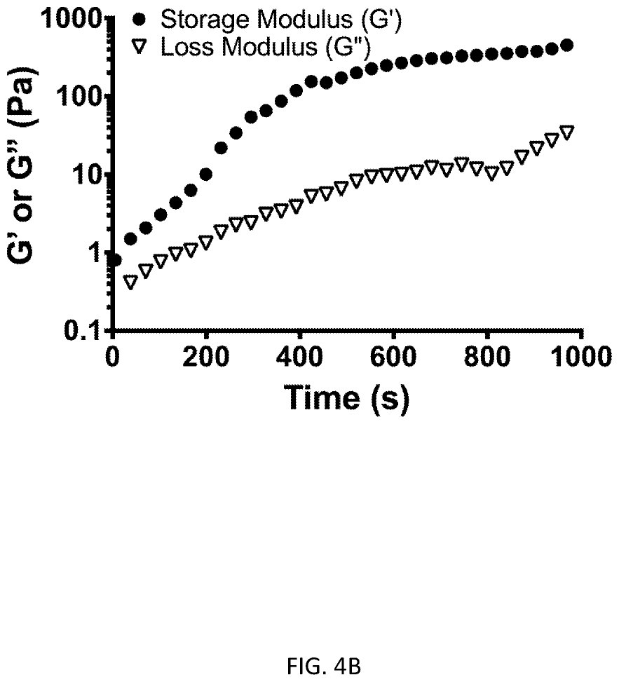

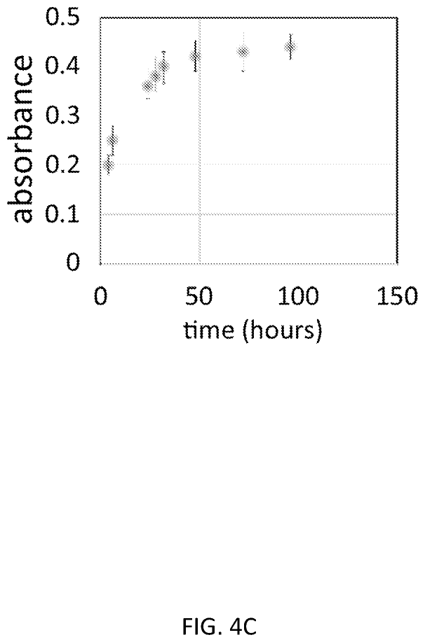

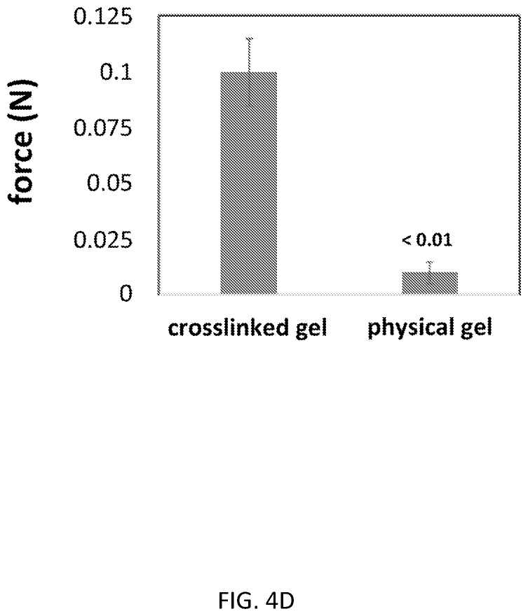

We have developed and evaluated in situ forming hydrogels composed of collagen type I (Vornia Biomaterials, UK) and multi-armed PEG-NHS linkers (JenKem Technologies), both of which have been previously used in commercially available medical implants. In situ gelation occurs in minutes under ambient conditions without the need for an external polymerization trigger (FIGS. 4A and 4B). A rheometer was used to track the substantial increase in modulus of the collagen upon mixing with 4-arm PEG-NHS (FIG. 4B). The gelation rates of these materials can be closely controlled by pH from seconds to many minutes, and in our preliminary work, these gels formed within 5 minutes at 37° C.

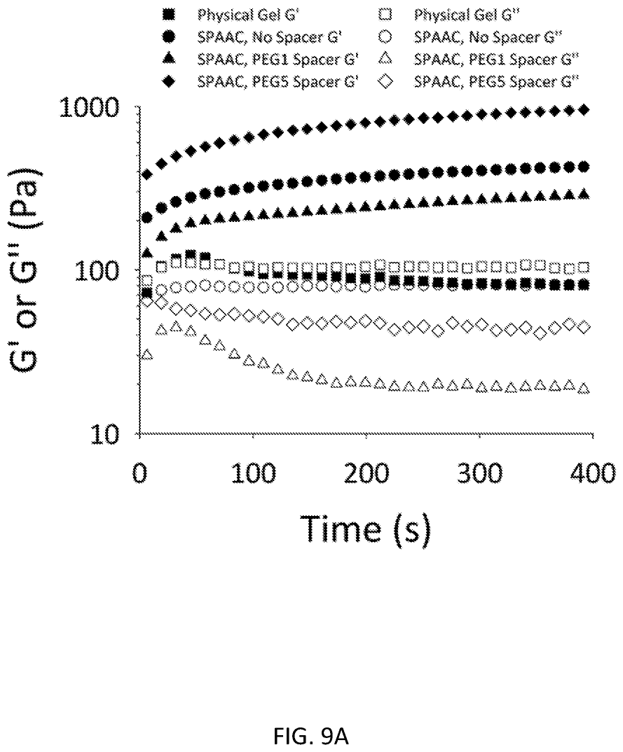

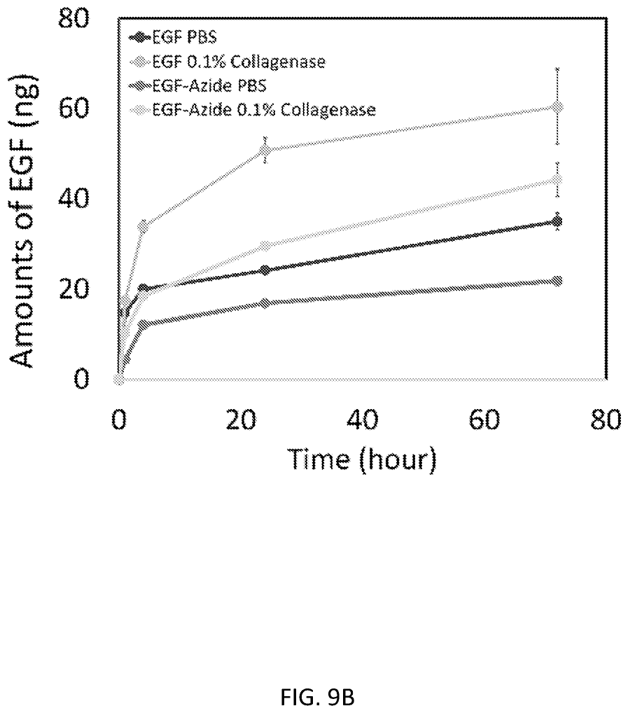

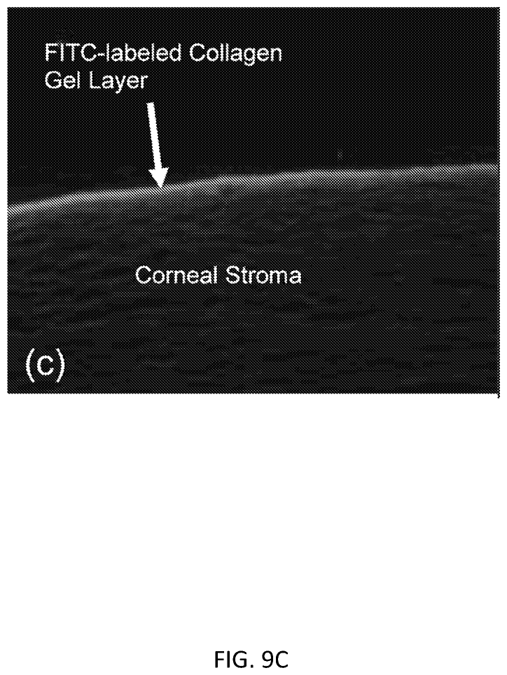

Azide-conjugated collagen was prepared by reaction of collagen with the heterobifunctional linker azide N-hydroxysuccinimide (NHS), and alkyne-functionalized collagen was prepared by reaction of collagen with the heterobifunctional crosslinker dibenzocyclooctyne-sulfo-N-hydroxysuccinimide (DBCO-sulfo-NHS). Varying spacer arm lengths were used for the azide conjugation: either PEG5, PEG1, or none (no spacer arm). The azide- and alkyne-functionalized conjugates were dialyzed and then mixed at room temperature. In situ gelation via SPAAC occurs rapidly under ambient conditions without the need for an external trigger (FIG. 9A). Rheometry was used to track the increase in the modulus of the gels upon crosslinking, which improved with the presence and length of the PEG spacer arm, compared to collagen solutions alone, which showed no change in their mechanical properties (FIG. 9A).

### EXAMPLE 4

### Growth Factor Release from SPAAC-Crosslinked Collagen Gels

Human recombinant EGF (Peprotech) was incorporated into collagen gels either physically (with unconjugated EGF), or by reaction with azide-conjugated EGF during the SPAAC reaction to form the collagen gel (using a slight excess of alkyne-functionalized collagen). The formed gels were then cut into cylindrical discs and placed in a PBS solution with or without 0.1% collagenase at 37° C. on a shaker. The solutions were then sampled at intervals starting at 2 hours to 96 hours. An ELISA kit was then used to evaluate the concentration of released EGF in the solution. The results, shown in FIG. 9B, indicate that with the current collagen gel formulations, a large proportion of the EGF is released rapidly over the first 6-8 hours followed by a slower release. The rate of release is increased with enzymatic degradation and reduced with covalent binding of EGF into the collagen gels via SPAAC. The data demonstrate the potential to control the release of EGF by chemically coupling it to the collagen matrix.

### EXAMPLE 5

### Adherence of Collagen-PEG Gels to Corneal Stroma

The NHS-crosslinked gels as well as physical collagen gels alone (without crosslinker) were cast between two debrided corneal explants cut to 100 mm² and allowed to gel between two stromal specimens for 15 minutes. Peel tests were done to determine the force required to delaminate the upper stromal specimen from the lower stromal specimen using a IN load cell. The crosslinked collagen gels showed a significant improvement in adhesion compared to physical collagen gels (FIG. 4D), which exhibits no chemical reactivity to stromal tissue collagen. The adhesion strength may be optimized by variation of the linker functionality (e.g., 4-arm versus 8-arm) as well as concentration.

### EXAMPLE 6

### Enhanced Cell Proliferation on Growth-Factor Fortified Collagen Gels

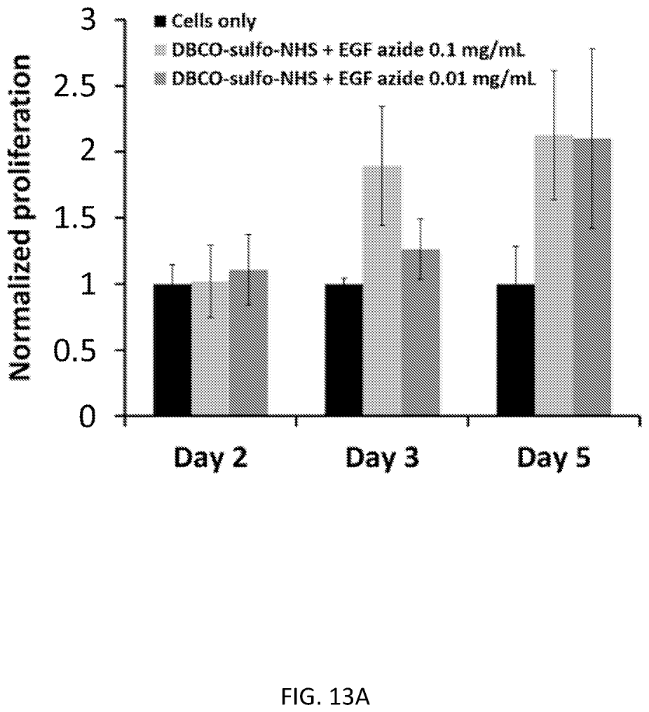

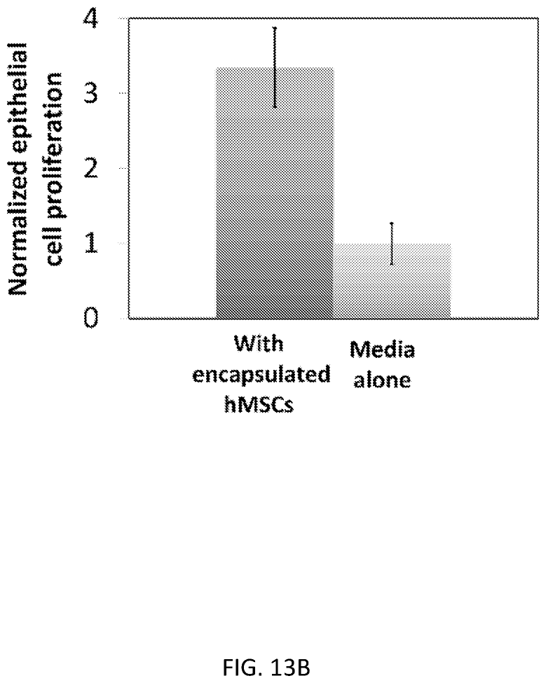

Late-passage primary rabbit corneal epithelial cells (CECs) of the same cell density were grown on surfaces with EGF bound to collagen using SPAAC click chemistry as well as standard collagen-coated tissue-culture polystyrene without covalently linked EGF. Greater proliferation of the otherwise slow-growing CECs was seen over the EGF-coupled collagen coatings over 5 days, with more rapid growth at 3 days in the case of higher EGF surface concentration (FIG. 13A), indicating that CEC adhesion and spreading are enhanced upon exposure to the bound growth factors in a dose-dependent manner. The results suggest that EGF chemically coupled to collagen remains bioactive and stimulates epithelial wound healing.

### EXAMPLE 7

### SPAAC Crosslinks Collagen Gels to Corneal Stroma

FITC-labelled collagen gels were crosslinked by SPAAC on ex vivo corneal stroma to evaluate its adhesion to corneal collagen. Briefly, FITC-labeled collagen was azide-functionalized while a second batch of alkyne-functionalized collagen was prepared. Porcine corneas were debrided over an 8 mm circular area and then treated with DBCO-sulfo-NHS for 10 minutes followed by rinsing with PBS. The FITC-labeled azide-collagen conjugate and alkyne-collagen conjugate were mixed on the surface of the cornea and quickly formed a gel that was adherent to the stroma. Debrided corneas were also treated with collagen-FITC solution alone for 10 minutes without prior alkyne-functionalization of the corneal stroma. The treated corneas were all irrigated aggressively with PBS, and then placed in 4% paraformaldehyde and processed for histological evaluation. The adherent, fluorescent gel is shown in cross-section in FIG. 9C.

### EXAMPLE 8

### Viability of Corneal Keratocytes and hMSCs Exposed to SPAAC and NHS Chemistry

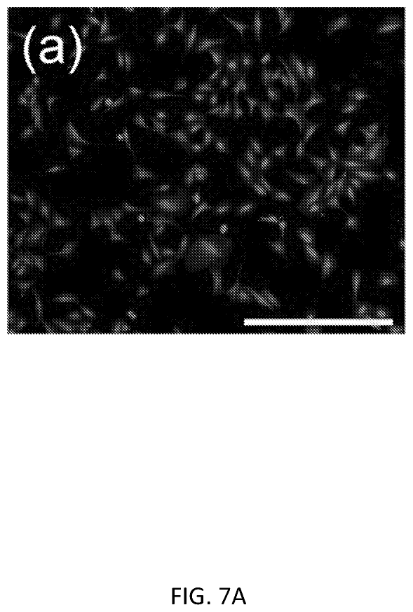

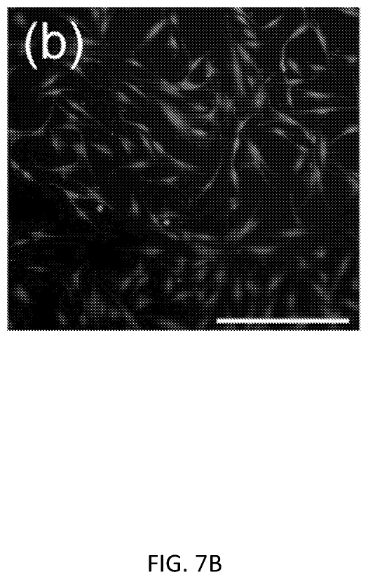

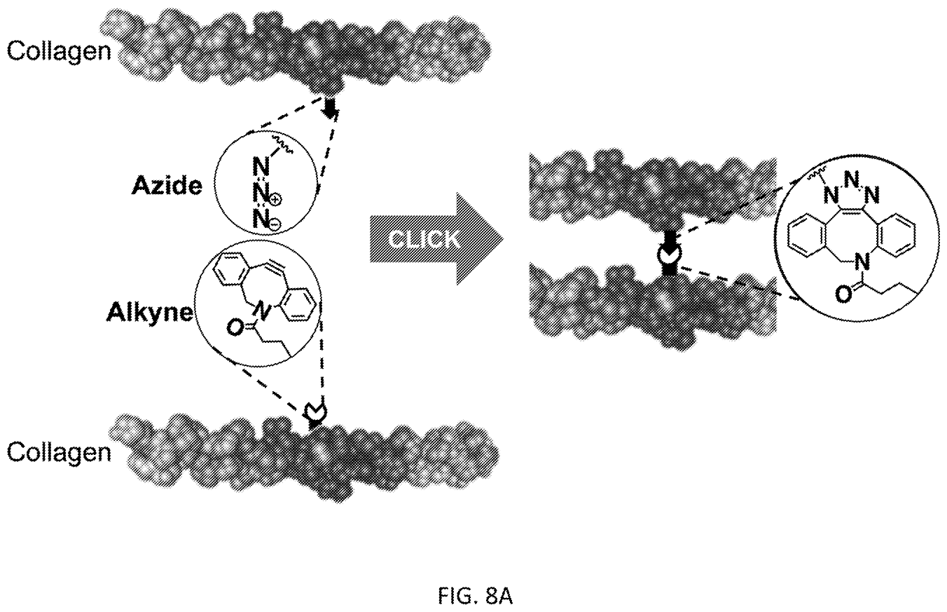

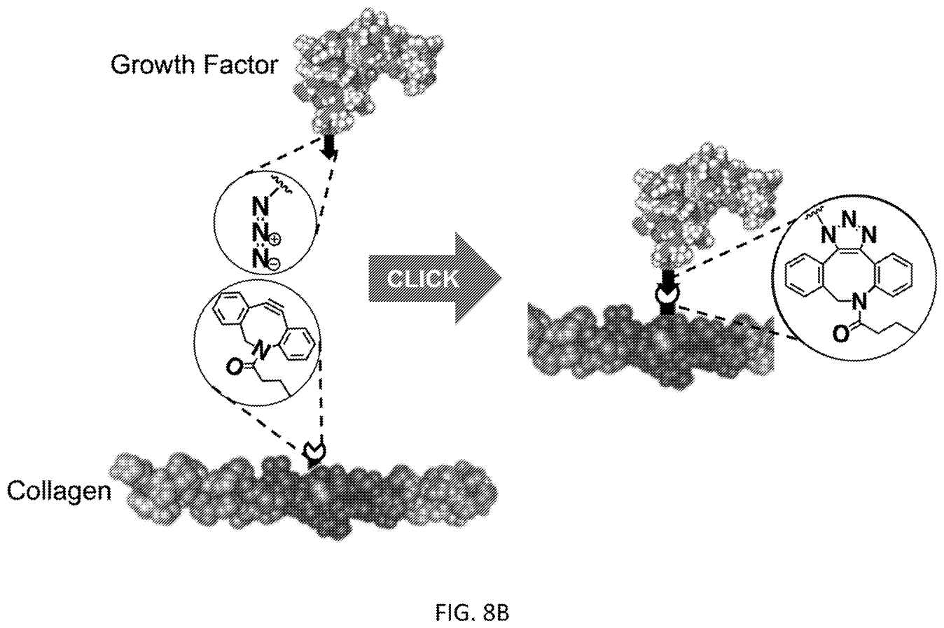

SPAAC click chemistry was performed in direct contact with a monolayer of primary rabbit corneal keratocytes, while collagen gels made by multi-functional NHS chemistry were used to encapsulate bone marrow-derived hMSCs. In the former experiment, keratocytes were directly exposed to 25 nM of dibenzocyclooctyne (DBCO)-sulfo-NHS (dissolved in PBS) followed by rinsing with sterile PBS and direct exposure to 100 nM azide-functionalized EGF in PBS. In the latter experiment, hMSCs were suspended within a fresh mixture of neutralized collagen (5 mg/mL) and then admixed with 4-arm PEG-NHS. A live-dead assay analysis showed that both the SPAAC click chemistry reaction and the NHS-mediated encapsulation were highly compatible with the exposed cells (FIGS. 7A and 7B). In both cases, cell viability was 97% or greater 72 hours after the chemical reaction. These results contrast with the control group, where pipette contact with a cell monolayer resulted in numerous dead cells. Taken together, these results bode well for the safety profile of applying these chemistries in situ to, for example wounded and neurotrophic corneas and other tissues.

### EXAMPLE 9

### SPAAC Click Chemistry for Binding Growth Factors to Collagen

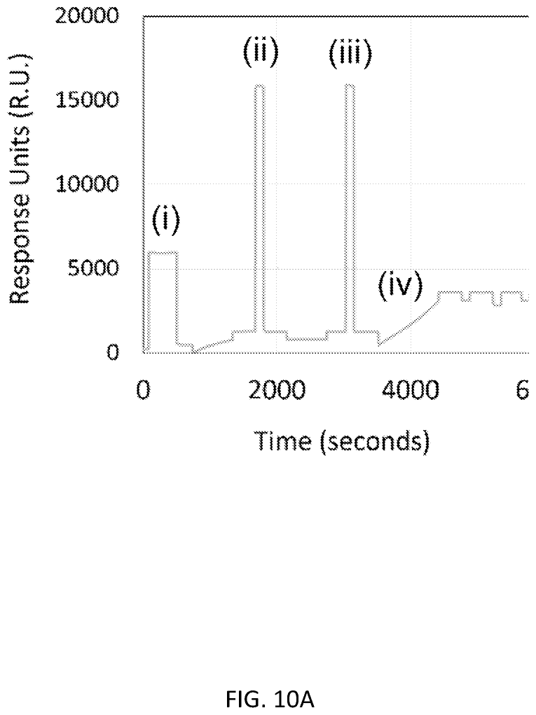

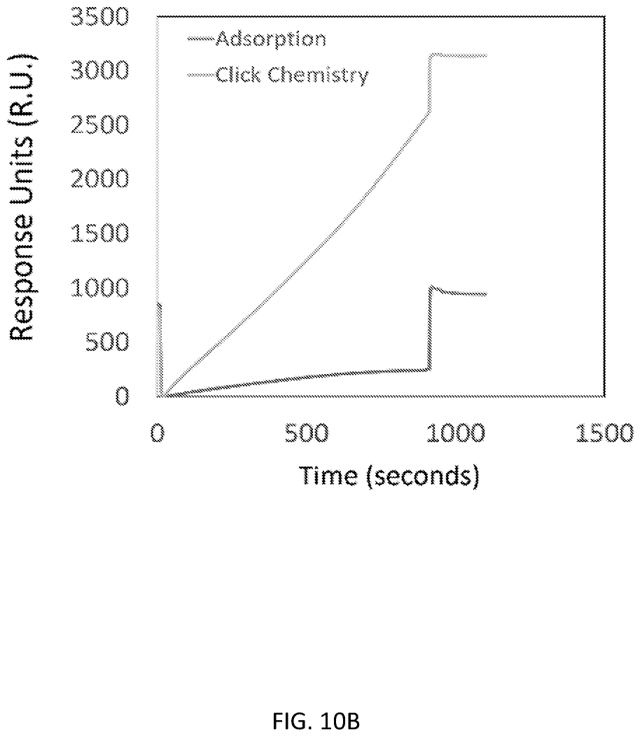

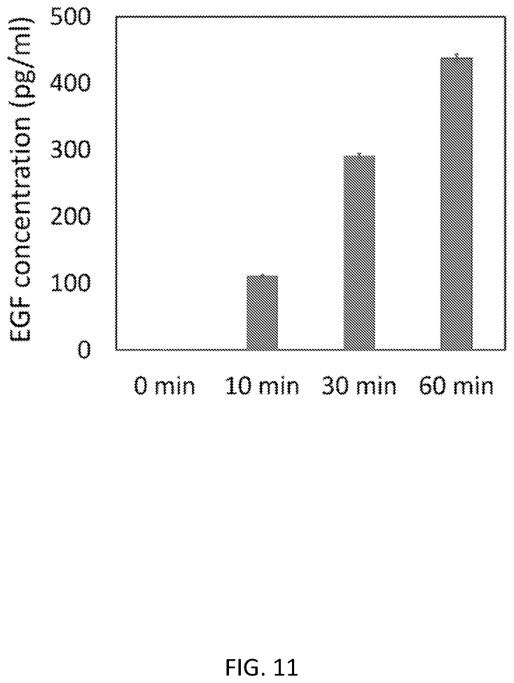

Surface Plasmon Resonance (SPR—FIGS. 10A and 10B), ellipsometry (Balevicius et al. (2014) Thin Solid Films 571:744-748; Kriechbaumer et al. (2012) PloS One 7(9):e46221, herein incorporated by reference), ELISA (FIG. 11), and X-ray photoelectron spectroscopy were used to monitor and compare the chemical coupling versus physical adsorption of growth factors to collagen surfaces. In these experiments, collagen was first chemically immobilized to either gold, glass, or polystyrene surfaces (depending on the method being used), followed by SPAAC-mediated coupling to the collagen or physical adsorption of growth factors from aqueous solution. Exemplary SPR results of NGF binding are shown in FIG. 10A and 10B which show increased real-time binding of NGF to collagen compared to physisorption. An ELISA assay (FIG. 11) showed an increase in the surface concentration of EGF bound to collagen as a function of reaction time (for a constant solution concentration of EGF-azide conjugate of 0.01 mg/mL).

### EXAMPLE 10

### Binding Growth Factors to Corneal Stroma with SPAAC Click Chemistry

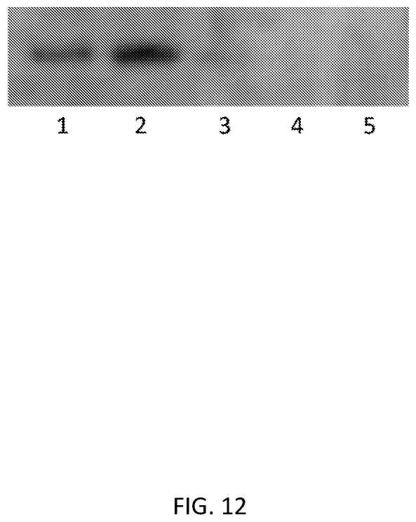

Western blot analysis was used to examine the binding of growth factors directly to corneal explant tissue (FIG. 2). EGF-FITC conjugates were bound to ex vivo porcine corneal stroma using photoactivation of riboflavin, no coupling (topical only), UV-phenyl azide coupling, and SPAAC click chemistry coupling. An anti-FITC antibody-horseradish peroxidase (HRP) conjugate was used to identify the presence or absence of the growth factor. The characteristic MW 6500 band was isolated in the click-coupling treatment, with only a faint band seen in the topical EGF case. Photochemical approaches showed evidence of coupling but also additional higher MW bands, indicative of the formation of crosslinked macromolecular EGF-collagen chimeric proteins that remain labeled with FITC. These macromolecular species do not dissociate due to direct carbon-carbon linkages that can occur as a result of the non-specific binding of light-based free-radical chemistry. Similar results were seen when using NGF (FIG. 12), where NGF-FITC was detected in greater proportion in corneal stroma with SPAAC relative to topical application alone for the same incubation time (10 minutes).

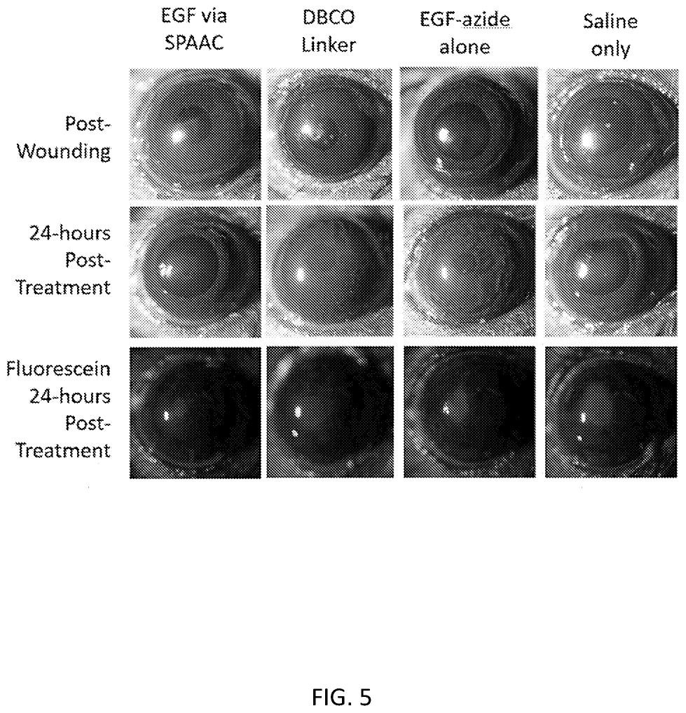

In addition, SPAAC was used to bind EGF to wounded corneas in a pilot animal study in rodents. Briefly, a 2 mm diameter debridement was performed, and the wound bed was treated topically with 25 nM DBCO-sulfo-NHS using a Weck-Cell, which was allowed to react for 10 minutes, followed by rinsing of the surface with balanced salt solution (BSS). This was followed by topical administration of 100 nM azide-functionalized EGF which was allowed to react for 10 minutes, and then rinsing with BSS. Controls included topically applied DBCO-sulfo-NHS linker alone, azide-functionalized EGF alone, and phosphate buffered saline (PBS). The eyes were examined clinically with a portable slit lamp and photographed at 1 hour, 24 hours, and 48 hours post-treatment. No signs of ocular intolerance such as conjunctival injection, swelling, discharge, or corneal toxicity were observed. Fluorescein staining at 24 hours (FIG. 5) revealed greater reduction in wound area in the EGF-coupled corneas compared with the DBCO linker, EGF-azide, and saline only treatments (n=2 for all treatments), with complete wound closure in all cases by 48 hours. Specifically, ImageJ analysis of the initial results showed that, on average (n=2) at 24 hours, the SPAAC-treated eyes had wounds that were approximately 40% of the area of the wounds observed in the topical EGF-treated eyes, and approximately 20% of the wound area of the untreated eyes. These results indicate that SPAAC is well-tolerated by the ocular surface, and that it has the potential to accelerate epithelial wound healing through the coupling of bioactive growth factors to corneal stroma.

### EXAMPLE 11

### Effect of Encapsulated hMSC-Secreted Factors

Primary CECs were plated in a 12-well plate and cultured in Dulbecco's modified eagle media without serum in the presence or absence of a transwell insert containing hMSCs encapsulated within a collagen gel. It was found that the proliferation of CECs was dramatically increased by the presence of the adjacent hMSCs within the collagen gels (FIG. 13B), indicating that the secreted factors emanating from the encapsulated hMSCs have a potent trophic effect.

### EXAMPLE 12

### Bioactivity of Conjugated Growth Factors

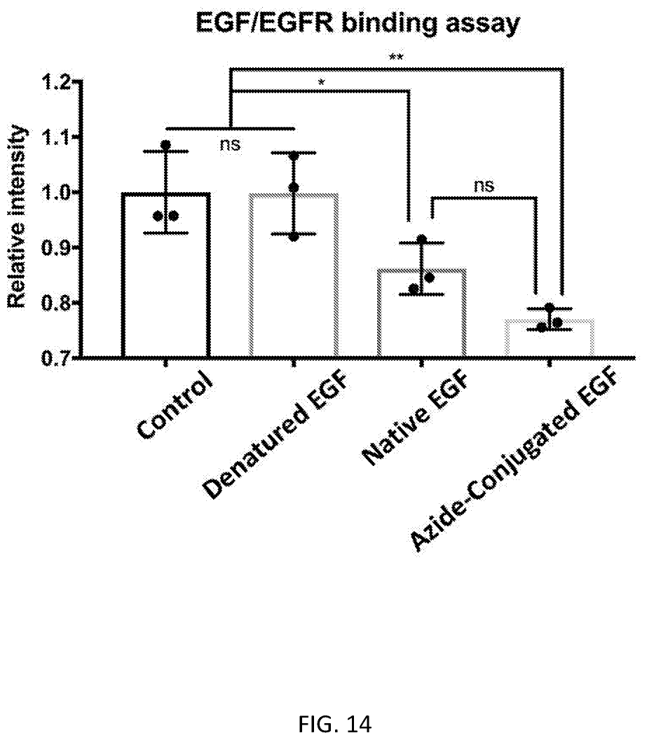

The bioactivity of EGF after conjugation (via NHS reaction to primary amines on EGF) was tested using an assay that quantifies competitive binding to the EGF receptor using a commercially available AlphaLISA kit. Bioactive but non-biotinylated EGF competes with biotinylated EGF for binding to the EGF receptor. Non-bioactive EGF will not bind to the EGF receptor. As shown in FIG. 14, denatured EGF does not reduce the baseline signal produced by the binding of biotinylated EGF with the EGF receptor, while both native EGF and azide-conjugated EGF do competitively bind the EGFR receptor (no statistically significant difference between native and conjugated EGF), indicating that EGF's bioactivity is preserved after conjugation with azide groups through reaction of its primary amines via N-hydroxysuccinimde chemistry.

While the preferred embodiments of the invention have been illustrated and described, it will be appreciated that various changes can be made therein without departing from the spirit and scope of the invention.

1. A method of treating damaged tissue in a subject, the method comprising:
a) contacting the damaged tissue with effective amounts of one or more growth factors capable of promoting tissue regeneration or repair; and
b) crosslinking the one or more growth factors to the damaged tissue using a biocompatible non-photochemical bioconjugation method.

2. The method of claim 1, wherein the one or more growth factors are selected from the group consisting of epidermal growth factor (EGF) and nerve growth factor (NGF).

3-4. (canceled)

5. The method of claim 2, wherein both EGF and NGF are crosslinked to the damaged tissue.

6. The method of claim 1, wherein said crosslinking comprises performing strain-promoted azide-alkyne cycloaddition (SPAAC) click chemistry or thiol-ene click chemistry.

7-16. (canceled)

17. The method of claim 1, wherein damage to the tissue is caused by physical trauma, chemical injury, surgery, or a disease.

18. The method of claim 1, wherein the damaged tissue is ocular tissue.

19. The method of claim 18, wherein the ocular tissue is corneal tissue or stromal tissue.

20-22. (canceled)

23. The method of claim 1, further comprising injecting reagents for the bioconjugation into a tissue subsurface.

24. The method of claim 1, wherein said crosslinking comprises performing more than one bioconjugation step.

25. The method of claim 1, further comprising performing at least one bioconjugation step with at least one of the one or more biomolecules in vitro prior to crosslinking said one or more biomolecules to the damaged tissue.

26. A method of treating damaged tissue in a subject, the method comprising:
a) providing a mixture comprising a hydrogel-forming molecule and at least one growth factor capable of promoting tissue regeneration or repair; and
b) forming a growth factor-eluting hydrogel in situ over the damaged tissue by using a biocompatible non-photochemical bioconjugation method to crosslink the hydrogel-forming molecule, wherein the hydrogel optionally encapsulates the at least one growth factor, and the hydrogel adheres to the treated tissue

27. The method of claim 26, wherein the hydrogel-forming molecule is selected from the group consisting of a glycoprotein, a carbohydrate, collagen, fibronectin, chitosan, laminin, hyaluronic acid, chondroitin sulfate, heparan sulfate, dermatan sulfate, chondroitin sulfate, polyethylene glycol, polyvinyl pyrrolidone, and polyvinyl alcohol.

28. The method of claim 27, wherein the collagen is collagen type I.

29. The method of claim 26, further comprising injecting the hydrogel forming agents into a tissue subsurface.

30. The method of claim 29, wherein the tissue subsurface is subcutaneous tissue or subconjunctival space.

31. The method of claim 26, wherein at least one growth factor is selected from the group consisting of epidermal growth factor (EGF) and nerve growth factor (NGF).

32. The method of claim 31, wherein both EGF and NGF are encapsulated in the hydrogel.

33. The method of claim 26, wherein the biocompatible non-photochemical bioconjugation method comprises performing strain-promoted azide-alkyne cycloaddition (SPAAC) click chemistry or thiol-ene click chemistry.

34-45. (canceled)

46. The method of claim 26, wherein damage to the tissue is caused by physical trauma, chemical injury, surgery, or a disease.

47. The method of claim 26, wherein the damaged tissue is ocular tissue.

48. The method of claim 47, wherein the ocular tissue is corneal tissue or stromal tissue.

49-50. (canceled)

51. A method of treating damaged tissue in a subject, the method comprising:
a) contacting the damaged tissue with a mixture comprising a hydrogel-forming molecule and stem cells; and
b) forming a hydrogel in situ on the damaged tissue by using a biocompatible non-photochemical bioconjugation method to crosslink the hydrogel-forming molecule, such that the hydrogel encapsulates the stem cells, wherein the encapsulated stem cells secrete growth factors that promote tissue regeneration or repair.

52. The method of claim 51, wherein the stem cells are mesenchymal stem cells.

53. The method of claim 51, wherein the stem cells are human stem cells.

54-56. (canceled)

57. The method of claim 51, wherein the hydrogel-forming molecule is selected from the group consisting of a glycoprotein, a carbohydrate, collagen, fibronectin, chitosan, elastin, laminin, hyaluronic acid, chondroitin sulfate, heparan sulfate, dermatan sulfate, chondroitin sulfate, polyethylene glycol, polyvinyl pyrrolidone, and polyvinyl alcohol.

58. The method of claim 57, wherein the collagen is collagen type I.

59. The method of claim 51, wherein the biocompatible non-photochemical bioconjugation method comprises performing strain-promoted azide-alkyne cycloaddition (SPAAC) click chemistry or thiol-ene click chemistry.

60-69. (canceled)

70. The method of claim 51, wherein damage to the tissue is caused by physical trauma, chemical injury, surgery, or a disease.

71. The method of claim 51, wherein the damaged tissue is ocular tissue.

72. The method of claim 71, wherein the ocular tissue is corneal tissue or stromal tissue.

73-75. (canceled)

76. The method of claim 51, further comprising encapsulating at least one growth factor in the hydrogel.
